(19) United States
(12) Patent Application Publication
(10) Pub. No.: US 2023/0183787 A1
(43) Pub. Date: Jun. 15, 2023

(54) RESTRICTION DIGEST BASED SEQUENTIAL DECODING

(71) Applicant: 10x Genomics, Inc., Pleasanton, CA (US)

(72) Inventors: Felice Alessio BAVA, Rome (IT); Toon VERHEYEN, Solna (SE)

(73) Assignee: 10x Genomics, Inc., Pleasanton, CA (US)

(21) Appl. No.: 18/079,681

(22) Filed: Dec. 12, 2022

Related U.S. Application Data
(60) Provisional application No. 63/265,349, filed on Dec. 13, 2021.

(51) Int. Cl. C12Q 1/6841; C12Q 1/682

(52) U.S. Cl. CPC C12Q 1/6841; C12Q 1/682

(57) ABSTRACT

The present application provides methods and compositions for analyzing biological samples involving sequential decoding of barcode regions of nucleic acid probes, wherein the barcode regions comprise barcode sequences that define a sequential signal code. In particular, the present application provides a method wherein probe hybridization generates a double-stranded recognition site for a nuclease. Cleavage of the double-stranded recognition site releases a sequence associated with the probe, thus releasing a detectable label associated with the probe.

### CROSS-REFERENCE TO RELATED APPLICATIONS

This application claims priority to U.S. Provisional Patent Application No. 63/265,349, filed on Dec. 13, 2021, entitled “RESTRICTION DIGEST BASED SEQUENTIAL DECODING,” which is herein incorporated by reference in its entirety for all purposes.

### FIELD

The present disclosure generally relates to methods and compositions for in situ detection of a plurality of molecules of one or more analytes in a sample.

### BACKGROUND

Nucleotide barcoding is used in a wide range of applications where it is desired to detect and differentiate multiple different analytes. Many barcoding methods use a limited pool of different detectable labels, and require multiple detection cycles in order to decode the barcode sequences. These methods typically involve repeatedly hybridizing a labelled detection probe, imaging the sample to detect the label that has been hybridized, and physically removing the hybridized labelled detection probe or detectable labels so that the next cycle can begin. In this way, a specified sequence of labels can be detected, forming a signal signature which distinguishes a particular analyte. Removing the hybridized labelled detection probe often involves the use of high temperature and/or potentially toxic chemical agents such as formamide to denature or disrupt the hybridization between the barcode and the detection probe. However, these methods are particularly problematic for in situ assays since they can damage the sample and interfere with desired downstream reactions. The present application addresses this and other needs.

### SUMMARY

In some aspects, provided herein is a method of analyzing a biological sample, comprising: a) contacting the biological sample with a nucleic acid probe, wherein the nucleic acid probe comprises a barcode region comprising one or more barcode sequences, and wherein the nucleic acid probe hybridizes to a target nucleic acid in the biological sample; b) hybridizing an oligonucleotide probe to a first hybridization region of the nucleic acid probe in the sample; wherein the first hybridization region comprises a first barcode sequence of the barcode region and a first single-stranded sequence, wherein hybridization of the oligonucleotide probe to the first single-stranded sequence creates a double stranded recognition site that is recognized by a nuclease, and wherein the first single-stranded sequence is not recognized by the nuclease; c) detecting a signal or absence thereof associated with the oligonucleotide probe at one or more locations in the biological sample; and d) cleaving the double stranded recognition site using the nuclease, thereby releasing a cleaved sequence associated with the oligonucleotide probe.

In some embodiments, the cleaved sequence comprises a first portion of the oligonucleotide probe hybridized to a first portion of the hybridization region. In some embodiments, a second portion of the oligonucleotide probe remains hybridized to a second portion of the hybridization region. In some embodiments, the method can comprise removing a second portion of the oligonucleotide probe. In some embodiments, the removing can comprise performing a wash step to remove the second portion of the oligonucleotide probe. In some embodiments, the wash step is performed using conditions that allow the oligonucleotide probe to remain hybridized to the nucleic acid probe.

In any of the preceding embodiments, the nucleic acid probe can be directly or indirectly hybridized to the target nucleic acid in the sample.

In any of the preceding embodiments, the nucleic acid probe can comprise a target binding region that hybridizes to the target nucleic acid in the sample.

In any of the preceding embodiments, the single-stranded sequence can be a spacer sequence positioned between the first barcode sequence and the rest of the nucleic acid probe, wherein the rest of the nucleic acid probe comprises the target binding region of the nucleic acid probe and/or one or more subsequent barcode sequences of the barcode region.

In any of the preceding embodiments, the oligonucleotide probe can be a first oligonucleotide probe and the nucleic acid probe can comprise a second hybridization region comprising a second barcode sequence of the barcode region, and the method can further comprise: contacting the biological sample with a second oligonucleotide probe comprising a sequence complementary to the second barcode sequence of the barcode region, wherein the second oligonucleotide probe hybridizes to the second hybridization region of the nucleic acid probe, and detecting a signal or absence thereof associated with the second oligonucleotide probe.

In some embodiments, the second hybridization region can comprise a second single-stranded sequence, wherein hybridization of the second oligonucleotide probe to the second single-stranded sequence creates a second double stranded recognition site that is recognized by a nuclease, and wherein the second single-stranded sequence is not recognized by the nuclease.

In any of the preceding embodiments, the first and second single-stranded sequences can have the same sequence. In some embodiments, the first and second single-stranded sequences can have different sequences.

In any of the preceding embodiments, the second single-stranded sequence can be a spacer sequence positioned between the second barcode sequence and the rest of the nucleic acid probe, wherein the rest of the nucleic acid probe comprises the target binding region of the nucleic acid probe and/or one or more subsequent barcodes.

In any of the preceding embodiments, the method can further comprise cleaving the second double-stranded recognition site using the nuclease, thereby releasing a cleaved sequence associated with the second oligonucleotide probe.

In some embodiments, the nucleic acid probe can further comprises a third hybridization region comprising a third barcode sequence of the barcode region, and the method can further comprise: contacting the biological sample with a third oligonucleotide probe comprising a sequence complementary to the third barcode sequence of the barcode region, wherein the third oligonucleotide probe hybridizes to the third hybridization region of the nucleic acid probe, and detecting a signal or absence thereof associated with the third oligonucleotide probe.

In some embodiments, the third hybridization region comprises a third single-stranded sequence, wherein hybridization of the third oligonucleotide probe to the third single-stranded sequence creates a third double stranded recognition site that is recognized by a nuclease, wherein the third single-stranded sequence is not recognized by the nuclease, and wherein the method comprises cleaving the third double-stranded recognition site using the nuclease, thereby releasing a cleaved sequence associated with the third oligonucleotide probe.

In any of the preceding embodiments, the first, second, and third single-stranded sequences can have the same sequence. In some embodiments, the first, second, and third single-stranded sequences can each have different sequences. In some embodiments, two of the first, second, and third single-stranded sequences can have the same sequence.

In any of the preceding embodiments, the nucleic acid probe can further comprise a fourth hybridization region comprising a fourth barcode sequence of the barcode region, and the method can further comprise: contacting the biological sample with a fourth oligonucleotide probe comprising a sequence complementary to the fourth barcode sequence of the barcode region, wherein the fourth oligonucleotide probe hybridizes to the fourth hybridization region of the nucleic acid probe, and detecting a signal or absence thereof associated with the fourth oligonucleotide probe.

In some embodiments, the fourth hybridization region can comprise a fourth single-stranded sequence, wherein hybridization of the fourth oligonucleotide probe to the fourth single-stranded sequence creates a fourth double stranded recognition site that is recognized by a nuclease, wherein the fourth single-stranded sequence is not recognized by the nuclease, and wherein the method comprises cleaving the fourth double-stranded recognition site using the nuclease, thereby releasing a cleaved sequence associated with the fourth oligonucleotide probe.

In any of the preceding embodiments, two or more of the first, second, third, and/or fourth single-stranded sequences can have the same sequence. In some embodiments, the first, second, third, and fourth single-stranded sequences can each have different sequences.

In any of the preceding embodiments, the second, third, and/or fourth single-stranded sequence can be a spacer sequence positioned between the second, third, or fourth barcode sequence, respectively, and the rest of the nucleic acid probe, wherein the rest of the nucleic acid probe comprises the target binding region of the nucleic acid probe and/or one or more subsequent barcode sequences.

In any of the preceding embodiments, the first barcode sequence can be selected from a first set of two, three, four, or more different first barcode sequences, and the method can comprise contacting the sample with a plurality of first oligonucleotide probes, wherein each first oligonucleotide probe comprises a sequence complementary to one of the first barcode sequences.

In any of the preceding embodiments, the second barcode sequence can be selected from a second set of two, three, four, or more different second barcode sequences, and the method can comprise contacting the sample with a plurality of second oligonucleotide probes, wherein each second oligonucleotide probe comprises a sequence complementary to one of the second barcode sequences.

In any of the preceding embodiments, the third barcode sequence can be selected from a third set of two, three, four, or more different third barcode sequences, and the method can comprise contacting the sample with a plurality of third oligonucleotide probes, wherein each third oligonucleotide probe comprises a sequence complementary to one of the third barcode sequences.

In any of the preceding embodiments, the fourth barcode sequence can be selected from a fourth set of two, three, four, or more different fourth barcode sequences, and the method can comprise contacting the sample with a plurality of fourth oligonucleotide probes, wherein each fourth oligonucleotide probe comprises a sequence complementary to one of the fourth barcode sequences.

In any of the preceding embodiments, each different barcode sequence within a given set can correspond to a different detectable label or absence thereof, wherein the label or absence thereof is directly or indirectly linked to the oligonucleotide probe that hybridizes to the barcode sequence.

In any of the preceding embodiments, the plurality of first oligonucleotide probes can be designed such that none of the first oligonucleotide probes hybridize to the any of the second, third, and/or fourth barcode sequences.

In any of the preceding embodiments, the plurality of second oligonucleotide probes can be designed such that none of the second oligonucleotide probes hybridize to any of the first, third, and/or fourth barcode sequences.

In any of the preceding embodiments, the plurality of third oligonucleotide probes can be designed such that none of the third oligonucleotide probes hybridize to any of the first, second, and/or fourth barcode sequences.

In any of the preceding embodiments, the plurality of fourth oligonucleotide probes can be designed such that none of the fourth oligonucleotide probes hybridize to any of the first, second, and/or third barcode sequences.

In any of the preceding embodiments, a sequential combination of the detected labels or absence thereof corresponding to the barcode sequences of the barcode region can form a sequential signal code that identifies the target nucleic acid hybridized by the nucleic acid probe.

In any of the preceding embodiments, each barcode sequence can be between 10 and 20 nucleotides in length.

In any of the preceding embodiments, the nucleic acid probe can be one of a plurality of nucleic acid probes, and the method can comprise contacting the biological sample with the plurality of nucleic acid probes.

In any of the preceding embodiments, the plurality of nucleic acid probes can comprise nucleic acid probes that hybridize to different target nucleic acids in the sample.

In any of the preceding embodiments, the plurality of nucleic acid probes can comprise a nucleic acid probe A that hybridizes to a target nucleic acid A and a nucleic acid probe B that hybridizes to a nucleic acid probe B.

In any of the preceding embodiments, the plurality of nucleic acid probes can comprises a group of nucleic acid probes A that hybridize to a target nucleic acid A and a group of nucleic acid probes B that hybridize to a target nucleic acid B.

In any of the preceding embodiments, the nucleic acid probe can further comprise an anchor binding region. In some embodiments, the anchor binding region can be a common sequence for the plurality of nucleic acid probes. In any of the preceding embodiments, the anchor binding region can be a common sequence among nucleic acid probes that hybridize to different target nucleic acids in the sample. In any of the preceding embodiments, the method can further comprise contacting the biological sample with an anchor probe, wherein the anchor probe hybridizes to the anchor binding region. In some embodiments, the method can comprise detecting a signal associated with the anchor probe hybridized to the anchor binding region.

In any of the preceding embodiments, from 5′ end to 3′ end or from 3′ end to 5′ end, the nucleic acid probe can comprise the first hybridization region, the second hybridization region, and the target binding region.

In any of the preceding embodiments, from 5′ end to 3′ end or from 3′ end to 5′ end, the nucleic acid probe can comprise the first hybridization region, the second hybridization region, the target binding region, and the anchor binding region.

In any of the preceding embodiments, from 5′ end to 3′ end or from 3′ end to 5′ end, the nucleic acid probe can comprise the first barcode sequence, the first single-stranded sequence, the second barcode sequence, the second single-stranded sequence, and the target binding region.

In any of the preceding embodiments, from 5′ end to 3′ end or from 3′ end to 5′ end, the nucleic acid probe can comprise the first barcode sequence, the first single-stranded sequence, the second barcode sequence, the second single-stranded sequence, the target binding region, and the anchor binding region.

In any of the preceding embodiments, the nucleic acid probe or each nucleic acid probe of the plurality of nucleic acid probes can comprise two, three, four, five, six, or more barcode sequences.

In any of the preceding embodiments, the nucleic acid probe or each nucleic acid probe of the plurality of nucleic acid probes can comprise two or more barcode sequences separated by the same single-stranded sequence.

In any of the preceding embodiments, each of the barcode sequences in the nucleic acid probe can be separated by the same single-stranded sequence. In any of the preceding embodiments, the nucleic acid probe can comprise two or more single-stranded sequences that each have different sequences. In any of the preceding embodiments, each of the single-stranded sequences in the nucleic acid probe can have different sequences.

In any of the preceding embodiments, the barcode region of the nucleic acid probe can be on one overhang region of the probe or can be on two overhang regions of the probe.

In any of the preceding embodiments, the hybridization regions can be ordered in the nucleic acid probe such that nuclease cleavage of a double-stranded recognition site created by hybridization of an oligonucleotide probe to a hybridization region does not release a subsequent hybridization region from the nucleic acid probe hybridized directly or indirectly to the target nucleic acid.

In any of the preceding embodiments, the method can comprise removing the cleaved sequence from the biological sample. In some embodiments, the removing can comprise a wash step.

In any of the preceding embodiments, the oligonucleotide probe can be directly associated with the label.

In any of the preceding embodiments, the oligonucleotide probe can be indirectly associated with the label by hybridization to one or more directly or indirectly detectably labeled probes.

In any of the preceding embodiments, the target nucleic acid or target nucleic acids comprise RNA. In any of the preceding embodiments, the target nucleic acid or target nucleic acids can comprise endogenous nucleic acids in the biological sample. In any of the preceding embodiments, the target nucleic acid or target nucleic acids can comprise mRNA.

In any of the preceding embodiments, the target nucleic acid can be a labelling agent associated with a target analyte in the biological sample.

In any of the preceding embodiments, the target nucleic acid can be a rolling circle amplification (RCA) product in the biological sample or in a matrix embedding the biological sample or molecules thereof.

In any of the preceding embodiments, the oligonucleotide probe can be associated with a plurality of copies of the label or with an absence of a label, thereby resulting in signal amplification of a signal detected from the label. In some embodiments, the signal amplification can comprise hybridization chain reaction (HCR) directly or indirectly on the oligonucleotide probe; linear oligonucleotide hybridization chain reaction (LO-HCR) directly or indirectly on the oligonucleotide probe; primer exchange reaction (PER) directly or indirectly on the oligonucleotide probe; assembly of branched structures directly or indirectly on the oligonucleotide probe; hybridization of a plurality of detectably labelled probes directly or indirectly on the oligonucleotide probe, or any combination thereof.

In some aspects, provided herein is a method of analyzing a biological sample, comprising: a) contacting the biological sample with a plurality of first oligonucleotide probes comprising first oligonucleotide probes A and B, wherein the biological sample comprises (i) a target nucleic acid A and a target nucleic acid B, and (ii) one or more nucleic acid probes A hybridized to the target nucleic acid A, and one or more nucleic acid probes B hybridized to the target nucleic acid B, wherein the one or more nucleic acid probes A comprise a barcode region A comprising a first barcode sequence selected from a set of first barcode sequences and a second barcode sequence selected from a set of second barcode sequences, wherein the one or more nucleic acid probes B comprise a barcode region B comprising a first barcode sequence selected from the set of first barcode sequences and a second barcode sequence selected from the set of second barcode sequences, and hybridization of the first oligonucleotide probes to the nucleic acid probes A and B creates double stranded recognition sites positioned between the first barcode sequences of barcode regions A and B and the rest of the nucleic acid probes A and B, respectively; b) at one or more locations in the biological sample, detecting a label or absence thereof associated with the oligonucleotide probes that hybridize to the first barcode sequence of barcode region A and the first barcode sequence of barcode region B; c) cleaving the double stranded recognition sites using a nuclease, thereby releasing a cleaved sequence associated with the first oligonucleotide probes A and B, respectively, leaving the second barcode sequences of barcode regions A and B in their respective nucleic acid probe; d) contacting the biological sample with a plurality of second oligonucleotide probes, wherein hybridization of the second oligonucleotide probes to the nucleic acid probes A and B creates double stranded recognition sites positioned between the second barcode sequences of barcode regions A and B and the rest of the nucleic acid probes A and B, respectively; e) at one or more locations in the biological sample, detecting a label or absence thereof associated with the oligonucleotide probes that hybridize to the second barcode sequences of barcode regions A and B; and f) using the labels or absence thereof detected in steps b) and e) to generate a sequential signal code corresponding to the target nucleic acid A and the target nucleic acid B, thereby identifying the target nucleic acids A and B at the one or more locations in the biological sample.

In some aspects, provided herein is a method of analyzing a biological sample, comprising: a) contacting the biological sample with a plurality of first oligonucleotide probes comprising first oligonucleotide probe A and first oligonucleotide probe B, wherein the biological sample comprises (i) a target nucleic acid A and a target nucleic acid B, and (ii) one or more nucleic acid probes A hybridized to the target nucleic acid A, and one or more nucleic acid probes B hybridized to the target nucleic acid B, wherein the one or more nucleic acid probes A comprise a barcode region A comprising a first barcode sequence selected from a set of first barcode sequences and a second barcode sequence selected from a set of second barcode sequences, wherein the one or more nucleic acid probes B comprise a barcode region B comprising a first barcode sequence selected from the set of first barcode sequences and a second barcode sequence selected from the set of second barcode sequences, wherein hybridization of the first oligonucleotide probes to the nucleic acid probe A creates a first double stranded recognition site positioned between the first barcode sequence of barcode region A and the rest of the nucleic acid probe A; and hybridization of the first oligonucleotide probes to the nucleic acid probe B creates a second double stranded recognition site positioned between the first barcode sequence of barcode region B and the rest of the nucleic acid probe B, and wherein the first double stranded recognition site is different from the second double stranded recognition site; b) at one or more locations in the biological sample, detecting a label or absence thereof associated with the oligonucleotide probes that hybridize to the first barcode sequence of barcode region A and the first barcode sequence of barcode region B; c) cleaving the first double stranded recognition site using a nuclease that cleaves the first double stranded recognition site but does not cleave the second double stranded recognition site, thereby releasing a cleaved sequence associated with the first oligonucleotide probe A, leaving the second barcode sequence of barcode region A in nucleic acid probe A, and leaving the first and second barcode sequences of barcode region B in nucleic acid probe B; d) at one or more locations in the biological sample, detecting a label or absence thereof associated with the oligonucleotide probes that hybridize to the first barcode sequences of barcode regions A and B; and e) using the labels or absence thereof detected in step b) and d) to generate a sequential signal code corresponding to the target nucleic acid A and the target nucleic acid B, thereby identifying the target nucleic acids A and B at the one or more locations in the biological sample.

In some embodiments, the first oligonucleotide probes can be designed such that none of the plurality of first oligonucleotide probes hybridize to the second barcode sequences.

In any of the preceding embodiments, the second oligonucleotide probes can be designed such that none of the plurality of second oligonucleotide probes hybridize to the first barcode sequences.

In any of the preceding embodiments, the one or more nucleic acid probes A and/or the one or more nucleic acid probes B can be directly hybridized to the nucleic acid A and nucleic acid B, respectively.

In any of the preceding embodiments, the one or more nucleic acid probes A and/or the one or more nucleic acid probes B can be indirectly hybridized to the first and second target nucleic acid sequences, respectively.

In any of the preceding embodiments, each of the nucleic acid probes A and B further can comprise an anchor binding region. In some embodiments, the anchor binding region can be a common sequence for the nucleic acid probes A and B. In any of the preceding embodiments, the method can further comprise contacting the biological sample with an anchor probe, wherein the anchor probe hybridizes to the anchor binding region, and detecting a signal associated with the anchor probe.

In any of the preceding embodiments, the first double stranded recognition site and the second double stranded recognition site can each comprise a recognition site that is cleaved by a different nuclease.

In any of the preceding embodiments, the nuclease is a restriction endonuclease.

In any of the preceding embodiments, the double-stranded recognition site can be 4, 5, 6, 7, 8, 9, 10, 11, 12, or more base pairs in length.

In any of the preceding embodiments, the double-stranded recognition site can be 6, 7, 8, or more base pairs in length.

In any of the preceding embodiments, cleavage of the double stranded restriction site can generate blunt ends.

In any of the preceding embodiments, the first double-stranded recognition site and/or the second double-stranded recognition site is 4, 5, 6, 7, 8, 9, 10, 11, 12, or more base pairs in length. In any of the preceding embodiments, the first double-stranded recognition site and/or the second double-stranded recognition site is 6, 7, 8, or more base pairs in length.

In any of the preceding embodiments, cleavage of the first double-stranded recognition site and/or the second double-stranded recognition site can generate blunt ends.

In some embodiments, the restriction endonuclease can be selected from the group consisting of anaI, Acc16I, AccII, AccBSI, AcvI, AfaI, AfeI, AjiI, AluBI, Aor51HI, BalI, BmgBI, Bsh1235I, BsnI, Bsp68I, BspANI, BspFNI, BsrBI, BssNAI, Bst1107I, BstSNI, BsuRI, BtuMI, DinI, DraI, Ecl136II, Eco105I, Eco147I, Eco321, Eco47III, Eco53kI, Eco721, EcoICRI, EcoRV, EgeI, EheI, FspI, HpaI, HpyCH4V, KspAI, MbiI, MluNI, Mox20I, MscI, Msp20I, MssI, MvnI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, and ZrmI.

In any of the preceding embodiments, cleavage of the double stranded restriction site can generate sticky ends.

In any of the preceding embodiments, cleavage of the first double-stranded recognition site and/or the second double-stranded recognition site can generate sticky ends.

In some embodiments the restriction endonuclease can be selected from the group consisting of AatII, AbsI, Acc65I, AccIII, AcII, AciI, AfIII, AgeI, AhII, Alw44I, Aor13HI, ApaI, ApaLI, AscI, AseI, AsiGI, AsiSI, Asp718I, AspA2I, AspLEI, AsuII, AvrII, BamHI, BauI, BbvCI, BcII, BcuI, BfaI, BfrI, BgIII, BlnI, BmgT120I, BmtI, Bpul4I, Bsa29I, BseAI, BseCI, BsePI, B seX3I, B seYI, B shTI, B shVI, B siWI, B spACI, B sp119, I, B sp120I, B spl3I, B sp1407I, B sp19I, BspDI, BspEI, BspHI, BspMAI, BspOI, BspT104I, BspTI, BsrGI, BssHII, BssMI, BssSI-v2, Bst2BI, BstAFI, BstAUI, BstBI, BstHHI, BstMBI, BstZI, Bsul5I, BsuTUI CciI, CciNI, CfoI, Cfr42I, Cfr9I, ClaI, CspAI, Csp6I, CviAII, CviQI, DpnII, EagI, EclXI, EcoRI, EcoT22I, FaeI, FatI, FauNDI, FbaI, FseI, GsaI, HapII, HhaI, HinIII, Hin6I, HindIII, HpaI, HpaII, HpySE526I, Hsp92II, KasI, Kpn2I, KpnI, Ksp22I, KspI, Kzo9I, MaeI, MboI, MluCI, MauBI, MfeI, MluI, Mlyl13I, Mph1103I, MreI, MroI, MroNI, MspCI, MseI, MspI, MunI, NarI, NcoI, NdeI, NdelI, NgoMIV, NheI, NlaIII, NotI, NsiI, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, RsaNI, SacI, SacII, SaII, SagAI, Sau3AI, SbfI, SdaI, SfaAI, Sfr274I, Sfr202I, SfuI, SgfI, SgrBI, SgrDI, SgsI, SlaI, SpeI, SphI, Sse83871, Sse9I, SsiI, SspDI, SspMI, SstI, TaiI, TaqI, TaqI-v2, TasI, TrulI, TspMI, Vha464I, VneI, VspI, XbaI, XhoI, XmaI, and Zsp2I.

In any of the preceding embodiments, the nuclease can be a nuclease that is capable of recognizing a double-stranded recognition site comprising one or more variable or degenerate positions. In some embodiments, the nuclease can be a restriction endonuclease selected from the group consisting of AasI, Acc36I, AccB7I, AcIWI, AdeI, AfiI, AhdI, Alw26I, AlwI, AlwNI, Asp700I, AspS9I, AxyI, BbsI, BccI, BcoDI, BfuAI, BgII, BlpI, Bme1390I, BmeRI, BmiI, BmrFI, BmrI, BmuI, BoxI, BpiI, Bpu10I, Bpu1102I, BsaBI, BsalI, Bsc4I, Bse21I, Bse8I, BseDI, BseGI, B selI, BseLI, BseMI, B sII, BsmAI, BsmBI-v2, BsmI, Bso31I, Bsp1720I, BspLI, BspMI, BspPI, BspQI, BspTNI, BsrDI, BsrI, BssECI, Bst4CI, Bst6I, BstAPI, BstC8I, BstEII, BstENI, BstF5I, BstMAI, BstMWI, BstPAI, BstPI, BstV2I, BstXI, Bsu36I, BtsCI, Btsl-v2, BtsIMutI, BveI, Cac8I, CaiI, Cfr13I, DdeI, DraIII, DrdI, DriI, DseDI, Eam1104I, Eam1105I, EarI, Eco31I, Eco81I, Eco91I, EcoNI, EcoO65I, Esp3I, FauI, Fnu4HI,Fsp4HI, HinfI, Hpyl66II, Hpyl88I, Hpyl88III, Hpy8I, HpyF10VI, HpyF3I, LguI, LmnI, MaeIII, MroXI, MsII, NIaIV, OliI, PaqCI, PciSI, PctI, PdmI, PflFI, PflMI, PfoI, PleI, PpsI, PshAI, PspEI, PspN4I, PspPI, PstNI, PsyI, RseI, SapI, ScrFI, SfiI, SmiMI, TaaI, Tth111I, Van91I, XagI, XcmI, and XmnI.

In some aspects, provided herein is a method of analyzing a biological sample, comprising: a) contacting the biological sample with a nucleic acid probe, wherein the nucleic acid probe comprises a barcode region comprising one or more barcode sequences, and wherein the nucleic acid probe hybridizes to a target nucleic acid in the biological sample; b) hybridizing a first oligonucleotide probe comprising a sequence complementary to a first barcode sequence of the barcode region to the first barcode sequence, c) hybridizing one or more detection probes to the first oligonucleotide probe, wherein the detection probes are associated with a label or with the absence of a label, wherein hybridization of the one or more detection probes to the first oligonucleotide probe creates one or more copies of a double stranded recognition site that is recognized by a nuclease; d) detecting a signal or absence thereof of the detection probes hybridized to the first oligonucleotide probe; e) cleaving the double-stranded recognition site using the nuclease, thereby releasing a cleaved sequence associated with the one or more detection probes. In some embodiments, the barcode sequence is not cleaved from the nucleic acid probe.

In some embodiments, the barcode region of the nucleic acid probe can comprise a second barcode sequence, and the method can further comprise: hybridizing a second oligonucleotide probe comprising a sequence complementary to the second barcode sequence of the barcode region to the second barcode sequence; hybridizing one or more detection probes to the second oligonucleotide probe; wherein the detection probes are associated with a label or with the absence of a label, wherein hybridization of the one or more detection probes to the second oligonucleotide probe creates one or more copies of a double stranded recognition site that is recognized by a nuclease, detecting the label or absence thereof of the detection probes hybridized to the second oligonucleotide probe; and cleaving the double stranded recognition site using the nuclease, thereby releasing a cleaved sequence associated with the one or more detection probes.

In any of the preceding embodiments, the biological sample can be non-homogenized and optionally selected from the group consisting of a formalin-fixed, paraffin-embedded (FFPE) sample, a frozen tissue sample, and a fresh tissue sample. In any of the preceding embodiments, the biological sample can be fixed. Alternatively, in any of the preceding embodiments, the biological sample may not be fixed. In any of the preceding embodiments, biological sample can be permeabilized. In any of the preceding embodiments, the biological sample can be embedded in a matrix, optionally wherein the matrix comprises a hydrogel. In any of the preceding embodiments, the biological sample can be cleared. In some embodiments, the clearing can comprise contacting the biological sample with a proteinase. In any of the preceding embodiments, the biological sample can be crosslinked. In any of the preceding embodiments, the biological sample can be a tissue slice between about 1 μm and about 50 μm in thickness, optionally wherein the tissue slice is between about 5 μm and about 35 μm in thickness.

In some aspects, provided herein is a kit for analyzing a biological sample, the kit comprising: (a) a plurality of nucleic acid probes, wherein each nucleic acid probe of the plurality comprises: (i) a target binding region complementary to a sequence of a target nucleic acid in the sample, and (ii) a barcode region comprising a first and second barcode sequence, wherein the first barcode sequence is selected from a set of first barcode sequences, and the second barcode sequence is selected from a set of second barcode sequences, wherein the first barcode sequence and second barcode sequence are separated by a single-stranded sequence; (b) a plurality of first oligonucleotide probes, wherein each first oligonucleotide probe of the plurality comprises a sequence complementary to one of the first barcode sequences of the set of first barcode sequences, and a sequence complementary to the single-stranded sequence; (c) a plurality of second oligonucleotide probes, wherein each second oligonucleotide probe of the plurality comprises a sequence complementary to one of the second barcode sequences of the set of second barcode sequences; and (d) a nuclease, wherein the nuclease does not recognize the single-stranded sequence, and the nuclease is capable of recognizing a double-stranded restriction site created by hybridization of a first oligonucleotide probe to the nucleic acid probe.

In some embodiments of the kit, the plurality of nucleic acid probes comprises a group of nucleic acid probes A comprising sequences complementary to sequences of a target nucleic acid A, and a group of nucleic acid probes B comprising sequences complementary to sequences of target nucleic acid B. In some embodiments, each nucleic acid probe of the plurality of nucleic acid probes comprises an anchor binding region. In some embodiments, the anchor binding region is a common sequence for the nucleic acid probes A and nucleic acid probes B. In some embodiments, the nuclease is a restriction endonuclease.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an exemplary method provided herein. A nucleic acid probe is hybridized to a target nucleic acid in a biological sample, wherein the nucleic acid probe comprises a barcode region comprising a first barcode sequence and a second barcode sequence (FIG. 1 (1)). A first oligonucleotide probe hybridizes to a first hybridization region comprising the first barcode sequence and a single-stranded sequence, wherein hybridization of the oligonucleotide probe to the hybridization region generates a double-stranded recognition site from the single-stranded sequence (FIG. 1 (2)). After detecting a label or absence thereof associated with the first oligonucleotide probe, the double-stranded recognition site is cleaved by a nuclease, thereby releasing a cleaved sequence associated with the first oligonucleotide probe (FIG. 1 (3)). As shown, the same single-stranded sequence can be used in a subsequent hybridization region (e.g., the second hybridization region, and the single-stranded sequence is not recognized by the nuclease. After cleavage of the first double-stranded recognition site, the rest of the nucleic acid probe (e.g., comprising one or more subsequent barcode sequences) can remain hybridized to the target nucleic acid (FIG. 1 (4)).

FIGS. 2A-2B schematically illustrate a method of analyzing a target nucleic acid using L-shaped oligonucleotide probes. FIG. 2A illustrates an L-shaped oligonucleotide probe hybridized to a first barcode sequence, thereby generating a double-stranded recognition site. Cleavage of the double-stranded recognition site by a nuclease releases a cleaved sequence associated with the L-shaped oligonucleotide probe. FIG. 2B shows an embodiment of an L-shaped oligonucleotide probe wherein hybridization of detection probes to overhang region of an oligonucleotide probe generates the double-stranded restriction endonuclease recognition site. Cleavage of the double-stranded recognition site by a nuclease releases a cleaved sequence associated with an overhang portion of the oligonucleotide probe, whereas the region of the oligonucleotide probe that is hybridized to a barcode sequence of the nucleic acid probe is not cleaved. In some embodiments, when the barcode sequence is not cleaved from the rest of the nucleic acid probes, the barcode sequences of the barcode region can be hybridized in any order.

FIG. 3 shows an exemplary workflow for restriction digest-based sequential decoding, where sequential hybridization and cleavage of barcodes in a first, second, third, and fourth cycle generates signature signals (for simplicity, the third cycle is not depicted). A group of nucleic acid probe(s) is hybridized to a given target nucleic acid sequence, wherein the nucleic acid probes comprise a barcode region that corresponds to the target nucleic acid. As shown, the barcode region can comprise a plurality of barcode sequences (BCs), wherein sequential detection of the barcode sequences defines a sequential signal code that identifies the target nucleic acid. The exemplary workflow of FIG. 3 can be used with any of the nucleic acid probes and oligonucleotide probes as shown in FIG. 1 and FIG. 2A-2B.

FIG. 4 depicts an exemplary method wherein hybridization of an oligonucleotide probe to a barcode sequence of a nucleic acid probe generates a double-stranded EcoRV recognition site. The barcode sequences are shown as a sequence of Ns, wherein N can be any nucleotide (A, T, G, or C). Cleavage of the double-stranded recognition site by EcoRV releases the first barcode sequence hybridized to a portion of the first oligonucleotide probe.

FIG. 5 shows an exemplary workflow for restriction digest-based sequential decoding, where restriction-based decoding is selective due to the nuclease selectively recognizing only some of the double-stranded recognition sites generated by hybridization of the oligonucleotide probe to the nucleic acid probe.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Single molecule fluorescent in situ hybridization (smFISH) is a widely used technique to determine expression levels by detecting mRNA. In smFISH, a set of typically 30-50 oligonucleotides, each about 20 nucleotides in length and each directly conjugated to a single fluorophore, are first hybridized to a complementary mRNA target. Individual transcripts are then visualized as diffraction-limited spots using wide-field epifluorescence microscopy, and quantified (Raj et al., 2008, Nat Methods, 5, 877-879). One limiting factor in this approach is that it is not good at multiplexing, (e.g., detecting more than 4 genes) as the multiplexing ability is limited by the number of fluorescent channels that are available. In order to identify more than 4 target nucleic acids (e.g., mRNAs), one would need to strip off the primary probes. After the probes are removed, primary probes targeting other target nucleic acids (e.g., mRNAs) can then be hybridized, etc. The process of removing the hybridized probe or detectable moiety after imaging can present challenges. This step often involves the use of high temperature and/or potentially toxic chemical agents such as formamide in the case of probe removal (stripping). In other embodiments such as MERFISH, fluorescent probes are bleached after imaging, and other fluorescently labelled probes are then able to bind a primary nucleic acid probe.

In some aspects, the present application provides an alternative approach for removing oligonucleotide probes associated with detectable labels after their hybridization and imaging, based on nuclease cleavage of double-stranded recognition sites. In some aspects, the double-stranded recognition site is generated by hybridization of the oligonucleotide probe to a hybridization region of a nucleic acid probe, wherein the hybridization region comprises a barcode sequence and a single-stranded sequence that is a single-stranded portion of the double-stranded recognition site. In some embodiments, the nuclease recognizes the double-stranded recognition site but does not recognize the single-stranded sequence. In this way, the same single-stranded sequence can be used between multiple barcode sequences of the nucleic acid probe, and the sequences will only be cleaved after hybridization of an oligonucleotide probe to said sequences. In some aspects, the sequences will only be cleaved after detection of the hybridized oligonucleotide probe to the sequences of the nucleic acid probe. Thus, oligonucleotide probes associated with detectable labels or the absence of a detectable label can be sequentially hybridized to a series of barcode sequences, and nuclease cleavage (e.g., restriction endonuclease cleavage) of the double-stranded recognition site generated by hybridization of the oligonucleotide probe can release a cleaved sequence associated with the oligonucleotide probe. The cleaved sequence can comprise or be linked to the detectable label associated with the oligonucleotide probe, thus allowing hybridization and detection of oligonucleotide probe associated with the same label in subsequent cycles. The present application thus provides an alternative decoding approach that reduces or eliminates the need for damaging probe stripping.

After image acquisition, the sequences of the oligonucleotide probes associated with detectable labels are removed by nuclease cleavage of the double-stranded recognition site in preparation for the next round of hybridization. The multiplex capacity scales as F^{N}, where F is the number of fluorophores (or the number of or the number of fluorophores plus one, if the absence of a detectable label is used as an additional “color” in the encoding/decoding scheme) and N is the number of rounds of hybridization. For example, the use of oligonucleotide probes associated with three distinguishable labels or with the absence of four distinct codes) n four rounds of sequential hybridization to four barcode sequences results in 4⁴, 256 potential distinct sequential signal codes. In some embodiments, not all possible sequential signal codes are used (e.g., depending on the error-correcting scheme used). Thus, in some aspects, decoding barcode regions comprising four barcode sequences using four distinguishable oligonucleotide probes is sufficient for detection of 200 or more distinct target nucleic acids. in some aspects, one can increase the multiplex capacity by increasing the number of rounds of hybridization with a limited pool of fluorophores.

In some aspects, provided herein is a method of analyzing a biological sample, comprising: contacting the biological sample with a nucleic acid probe, wherein the nucleic acid probe comprises a barcode region comprising one or more barcode sequences, and wherein the nucleic acid probe hybridizes to a target nucleic acid in the biological sample; hybridizing an oligonucleotide probe to a first hybridization region of the nucleic acid probe in the sample; wherein the first hybridization region comprises a first barcode sequence of the barcode region and a first single-stranded sequence, wherein hybridization of the oligonucleotide probe to the first single-stranded sequence creates a double stranded recognition site that is recognized by a nuclease, and wherein the first single-stranded sequence is not recognized by the nuclease; detecting a signal or absence thereof associated with the oligonucleotide probe; and cleaving the double stranded recognition site using the nuclease, thereby releasing a cleaved sequence associated with the oligonucleotide probe. In some embodiments, the same single-stranded sequence can be used between multiple barcode sequences of the nucleic acid probe, because the single-stranded portion of the double-stranded recognition site is not recognized by the nuclease (e.g., restriction endonuclease). Thus, sequential hybridization of oligonucleotide probes to sequential barcode sequences of the nucleic acid probe allows specific cleavage of only the barcode sequence hybridized by the oligonucleotide probe in each cycle. The barcode sequences can be arranged such that barcode sequences detected in earlier cycles are closer to an end of the nucleic acid probe, whereby only the detected barcode sequence is cleaved from the probe in each cycle.

In some embodiments, the present disclosure provides a U-probe design of a nucleic acid probe. The nucleic acid probe can be a primary probe that hybridizes to a target mRNA in the sample. The nucleic acid probe can comprise a target-binding region (e.g., a 20-30 bp target binding region), an overhang comprising an anchor binding region to bind an anchor probe (e.g., an detectably labelled anchor probe, and a second overhang comprising a barcode region, wherein the barcode region comprises four barcode sequences interspersed with single-stranded sequences that are single-stranded portions of double-stranded recognition sites for one or more selected nucleases. The combination of the barcode sequences in the barcode region can encode for a specific gene. In an example, nucleic acid probes are designed to detect each barcode sequence (e.g., of four barcode sequences in the barcode region) using one of four different oligonucleotide probes, resulting in a coding capacity of 4⁴=256. In an example, nucleic acid probes for about 200 genes are hybridized to the target mRNAs (e.g., about 20-50 nucleic acid probes per mRNA), and a plurality of oligonucleotide probes designed to bind to the first barcode sequences and first single-stranded sequences are allowed to hybridize to the nucleic acid probes. The sample can then be imaged. As the oligonucleotide probes also hybridize to the first single-stranded sequence, the single-stranded sequence becomes the double-stranded recognition site and can be recognized by a nuclease (e.g., a restriction endonuclease).

In various embodiments, the double-stranded recognition site can be a 4 bp, 6 bp, or 8 bp sequence. After imaging, a restriction digestion reaction can performed and a sequence associated with the oligonucleotide probe as well as the first barcode sequence of the nucleic acid probe are cleaved off. Cleavage of the double-stranded recognition site can result in blunt ends or in sticky ends. In some embodiments, the cleavage leaves a second portion of the oligonucleotide probe hybridized to the nucleic acid probe (e.g., for a 6 bp double-stranded recognition site, a 3 nucleotide portion of the oligonucleotide probe may remain hybridized to the nucleic acid probe). This short fragment can remain bound or be washed away in a separate wash step or during hybridization of sequencing probes to the second barcode. This process of oligonucleotide probe hybridization, detection (e.g., imaging), and nuclease cleavage can be repeated until all barcode sequences of the nucleic acid probe have been used. In some embodiments, an anchor probe can be hybridized to the anchor binding region (e.g., a common sequence) found on the first overhang of the nucleic acid probe to visualize nucleic acid probes. This system thus allows for the simultaneous detection and decoding of 200 or more mRNA targets within a tissue section without the need for rehybridizing the primary probe. The approaches described herein can be coupled with any decoding and/or error-correcting method described herein to boost the number of mRNA targets that can be decoded in a small number of cycles.

Various aspects of the methods and compositions provided herein are described in further detail in the sections below. Section II describes various samples, analytes, and target sequences that can be analyzed using the methods provided herein. Section III describes probes and probe designs for use in the methods described herein, such as the nucleic acid probes and oligonucleotide probes described above. Section IV describes methods of nuclease digest-based sequential decoding. Section V describes aspects of signal amplification, detection, and analysis provided herein. Kits for use in the methods described herein are provided in Section VI.

As noted above, the section headings used herein are for organizational purposes only and are not to be construed as limiting the subject matter described.

### II. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stooI, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanoI, methanoI, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes. In some embodiments, one or more post-fixing step is performed after a hybridization complex comprising a probe and a target is formed in a sample.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer materiaI, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample can be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015, the content of which is herein incorporated by reference in its entirety. In some embodiments, isometric expansion can be performed as described in US20190276881 and in Wang et al. Scientific reports vol. 8,1 4847. 19 Mar. 2018, the contents of each of which is herein incorporated by reference in its entirety.

Isometric expansion can be performed by anchoring one or more components of a biological sample to a geI, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In generaI, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible cros slinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycoI, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid materiaI, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogeI, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In generaI, a biological sample can be permeabilized by exposing the sample to one or more permeabilizing agents. Suitable agents for this purpose include, but are not limited to, organic solvents (e.g., acetone, ethanoI, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al., Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosoI, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte). Alternatively, the specific binding partner may be coupled to a nucleic acid, which may be detected.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channeI, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte (e.g., a target nucleic acid) comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence of an endogenous mRNA, or sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a nucleic acid probe comprising a barcode region as described herein), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channeI, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA, such as an mRNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed using the nucleic acid probes, oligonucleotide probes, and methods described herein. In some embodiments, a labelling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labelling agent that directly or indirectly binds to an analyte in the biological sample is analyzed.

(a) Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent (e.g., reporter oligonucleotide attached thereto). The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In some embodiments, the nucleic acid probes comprising barcode regions for described herein can be hybridized to a primary probe, wherein the primary probe is hybridized to a target analyte in the sample. In some embodiments, the nucleic acid probes comprising barcode regions for described herein can be hybridized directly to an endogenous target analyte in the sample.

Various probes and probe sets can be hybridized to an endogenous analyte and/or a labelling agent and each probe may comprise one or more barcode sequences. In some embodiments, the nucleic acid probes comprising barcode regions described herein can be hybridized to an amplification product such as a rolling circle amplification (RCA) product generated from a probe or probe set in the sample. In some embodiments, the use of nucleic acid probes described herein for sequential decoding can reduce the length requirement of a barcoded probe or probe set (e.g., by allowing sequential decoding of a plurality of barcode sequences comprised by the nucleic acid probe, thus expanding the coding capacity without adding additional barcode sequence to the probe or probe set for generating the RCA product). Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design analyzed using the nucleic acid probes and methods described herein can vary.

(b) Ligation

In some embodiments, a product of an endogenous analyte and/or a labelling agent is a ligation product. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labelling agent (e.g., the reporter oligonucleotide) or a product thereof.

In some embodiments, a nucleic acid probe described herein hybridizes to a product of a probe or probe set capable of DNA-templated ligation, such as from a cDNA molecule. See, e.g., U.S. Pat. 8,551,710, which is hereby incorporated by reference in its entirety. In some embodiments, the probe or probe is set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, the nucleic acid probes and decoding methods described herein can be applied in a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product analyzed using the nucleic acid probes and methods described herein is a primer extension product of an analyte, a labelling agent, a probe or probe set bound to the analyte (e.g., a padlock probe bound to genomic DNA, mRNA, or cDNA), or a probe or probe set bound to the labelling agent (e.g., a padlock probe bound to one or more reporter oligonucleotides from the same or different labelling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of 0appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) may include linear RCA, a branched RCA, a dendritic RCA, or any combination thereof. (See, e.g., Baner et al., Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et aI, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N- hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays can be used for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a nucleic acid probe disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe (e.g., a nucleic acid probe described herein). The exogenously added nucleic acid probe may be optionally ligated to a cellular nucleic acid molecule or another exogenous nucleic acid molecule. In other examples, a product comprising a target sequence for a nucleic acid probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a nucleic acid probe disclosed herein may a probe hybridizing to an RCP. The probe may comprise an overhang that does not hybridize to the RCP but hybridizes to another probe (e.g., a nucleic acid probe described herein). The probe may be optionally ligated to a cellular nucleic acid molecule or another probe, e.g., an anchor probe that hybridize to the RCP.

C. Target Sequences

A target sequence (e.g., in a target nucleic acid) for a probe disclosed herein (e.g., a nucleic acid probe described herein) may be comprised in any analyte disclose herein, including an endogenous analyte (e.g., a viral or cellular nucleic acid), a labelling agent, or a product of an endogenous analyte and/or a labelling agent.

In some embodiments, a target sequence for a nucleic acid probe described herein is a marker sequence for a given analyte. A marker sequence is a sequence that identifies a given analyte. The marker sequence for a given target analyte must therefore be specific to that analyte, or unique, such that multiple target analytes can be distinguished from each other.

A “marker sequence” is thus a sequence which marks, is associated with, or identifies a given analyte. It is a sequence by which a given analyte may be detected and distinguished from other analytes. Where an “analyte” comprises a group of related molecules e.g. isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g. a particular variant. In this way different variants, or isoforms, or mutants may be identified or distinguished from one another.

Where the target analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be incorporated into the target nucleic acid molecule as a tag or identifier (ID) sequence (e.g. a barcode) for the analyte (including for a nucleic acid analyte). It may thus be a synthetic or artificial sequence.

Where the target nucleic acid molecule is generated from a target analyte or as a reporter for said analyte, the target sequence (e.g., a marker sequence) may be a complementary copy of a sequence present in a template which is used to generate the target nucleic acid molecule, for example, in a probe or a part thereof, e.g. where the target nucleic acid molecule is an amplification product, it may be a complementary to a sequence present in the template which is amplified. In an embodiment, the target sequence (e.g., a marker sequence) may be a complementary copy of a sequence present in an RCA template, where the target nucleic acid molecule is an RCP. The RCA template may be part of a probe, or may be generated or provided in the assay method, for example by circularization of a linear probe or probe component.

It can be seen that where the target nucleic acid molecule is generated directly from a target nucleic acid analyte then, again, the target sequence (e.g., a marker sequence) may be the complement of a sequence present in the target analyte molecule. However, where the target nucleic acid molecule is generated from an alternative template (examples of which are set out below), the target sequence (e.g., a marker sequence) may be the complement of a sequence present in said template. The complement of the target sequence may thus be provided in the template for producing the target nucleic acid molecule as a tag or identifier sequence for the analyte, for example where the template for the target nucleic acid molecule is or is generated from a probe (e.g. a circularizable probe such as a padlock probe), or where the template for the target nucleic acid molecule is a reporter for the analyte (e.g. in an immunoRCA reaction). It will be understood in this regard that the sequence in the template which is complementary to the target sequence (e.g., a marker sequence) present in the target nucleic acid molecule may itself be regarded as a marker sequence. The template may be provided or generated from a probe or reporter molecule which is designed to detect a particular analyte, and thus such a probe or reporter molecule may be viewed as comprising a marker sequence for that analyte—the marker sequence is then copied, as a complementary sequence, into the target nucleic acid molecule. The term “marker sequence” can therefore encompass both a marker sequence present in the target nucleic acid molecule and its complement (more particularly reverse complement) present in the template for the target nucleic acid molecule. Accordingly, a “marker sequence” can include the complementary sequence.

Similarly, each target nucleic acid molecule may comprise multiple copies of the target sequence (e.g., a marker sequence). Thus a probe molecule, or probe component, including a padlock probe as described above, may comprise multiple copies of a target sequence (e.g., a marker sequence). In another example, an amplification product may be generated which comprises multiple copies of the target sequence (e.g., a marker sequence). In an embodiment, where the target nucleic acid molecule is an RCP, i.e. a concatemer of monomer repeats produced by repeated amplification of a circular template, the target nucleic acid molecule will comprise a plurality of target sequence (e.g., a marker sequence). Accordingly, when the target nucleic acid molecule comprises multiple copies of the marker sequence, multiple HCR initiators will be comprised within, or can be hybridized to, each target nucleic acid molecule. In turn, this means that multiple HCR reaction can be initiated, and multiple HCR products can be generated, from a single target analyte.

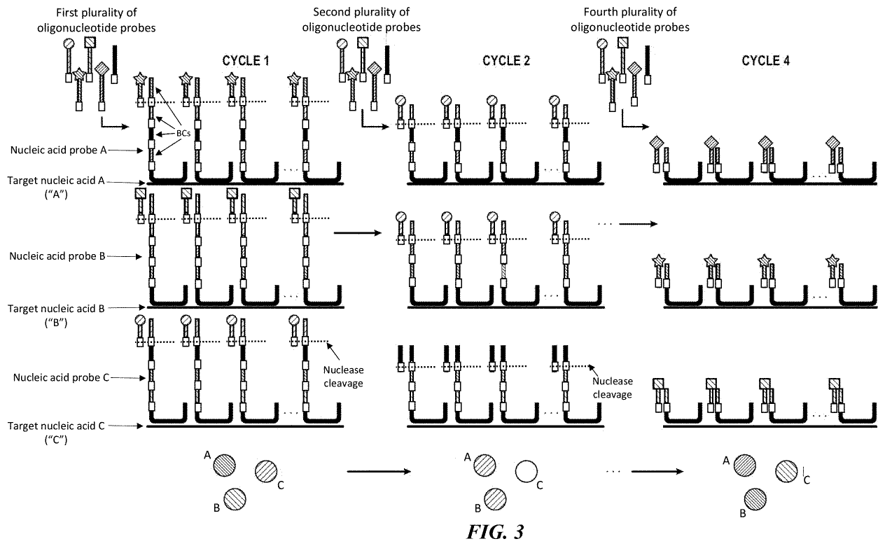

In some embodiments, a group of nucleic acid probes hybridize to a group of target sequences in a target nucleic acid molecule, such as an RNA (e.g., mRNA). For example, a group of nucleic acid probes described herein can be designed to hybridize to a plurality of target sequences (e.g., marker sequences) present in the nucleic acid molecule. In some embodiments, the target nucleic acid comprises between or between about 10 and 20, 10 and 15, 10 and 30, 20 and 30, 20 and 40, 20 and 50, 40 and 50, or 45 and 60 target sequences (e.g., marker sequences). The target sequences can comprise copies of the same target sequences and/or can comprise different target sequences. Hybridization of a plurality of nucleic acid probes to a given target nucleic acid increases the number of binding sites available for oligonucleotide probes associated with labels or with the absence of a label, resulting in a strong signal intensity, and thus an increase in the signal to noise ratio. In turn, this allows for highly sensitive detection of target analytes, and enables, for example, the detection of rare transcripts or mutations. In some embodiments, a plurality of nucleic acid probes that hybridizes to each given target nucleic acid shares the same barcode sequences (see e.g., FIG. 3).

In some embodiments, target sequences in a target nucleic acid molecule such as an RNA (e.g., mRNA) are designed to optimize hybridization of a plurality of nucleic acid probes under a constant set of hybridization conditions, e.g., incubation temperature. For example, target sequences can be designed to cover a relatively narrow range of GC content and melting temperatures (TM) with the target binding regions of the corresponding nucleic acid probes. In some embodiments, target sequences for endogenous RNAs have limited homology to other RNAs in the transcriptome, reducing the probability that a nucleic acid probe will bind to the wrong RNA. Design considerations for target sequences have been described, for example, in U.S. Patent Application Publication Nos. 2017/0220733 and 2017/0212986; and U.S. Pat. No. 11,098,303, the contents of each of which are herein incorporated by reference in their entirety.

It will be understood that in the case of a target nucleic acid molecule comprising multiple target sequences (e.g. marker sequences), while each of the target sequences (e.g. marker sequences) comprises a binding site for a nucleic acid probe described herein, in practice not all of these binding sites may (or will) be occupied by a nucleic acid probe after nucleic acid probe hybridization. It suffices that a number, or multiplicity, of such binding sites are bound by a nucleic acid probe. Thus, in some embodiments the nucleic acid probe may hybridize to at least one target sequence (e.g., marker sequence) present in a target nucleic acid molecule. In some embodiments, the nucleic acid probe hybridizes to multiple target sequences present in the target nucleic acid molecule.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

### III. Probes

Disclosed herein in some aspects are nucleic acid probes, oligonucleotide probes, and anchor probes, that are introduced into a cell or used to otherwise contact a biological sample such as a tissue sample. The probes may comprise any of a variety of entities that can hybridize to a nucleic acid, typically by Watson-Crick base pairing, such as DNA, RNA, LNA, PNA, etc. The probes typically contain a targeting sequence or hybridization region that is able to directly or indirectly bind to at least a portion of a target nucleic acid or a probe. For example, nucleic acid probes described herein may be able to bind to a specific target nucleic acid (e.g., an mRNA, or other nucleic acids as discussed herein). In some embodiments, the nucleic acid probes may be detected using oligonucleotide probes that bind to the nucleic acid probes. In some embodiments, the oligonucleotide probes are directly or indirectly associated with a detectable label. In some embodiments, oligonucleotide probes are indirectly associated with a detectable label by binding of one or more detection probes comprising detectable labels to the oligonucleotide probes. In some embodiments, the oligonucleotide probes are directly or indirectly detectable in situ (e.g., at a location in the biological sample).

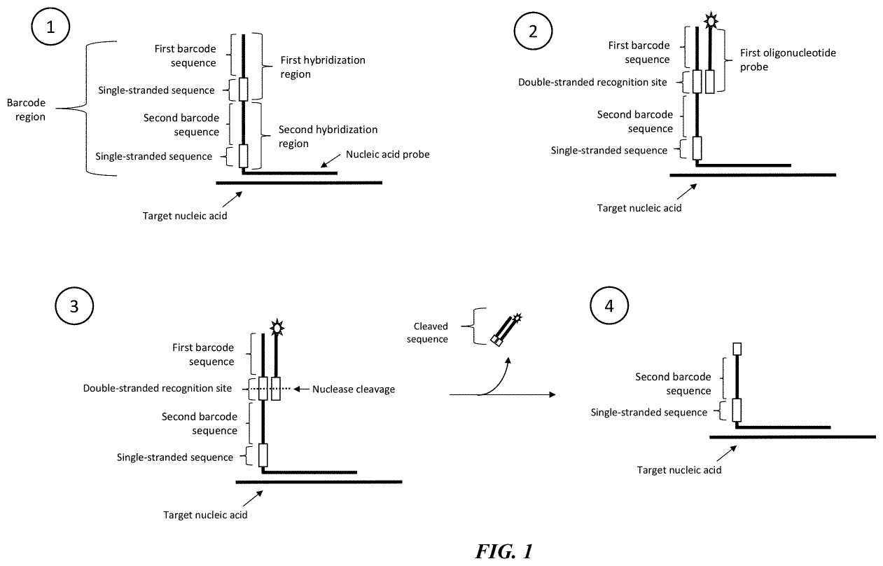

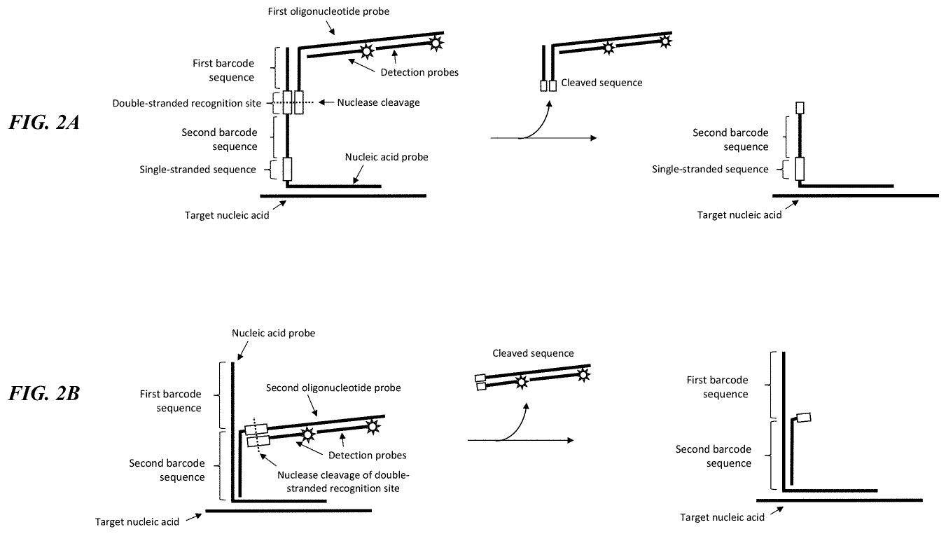

In some embodiments, the probes (e.g., nucleic acid probes and/or oligonucleotide probes) comprise single-stranded sequences that are not recognized by a given nuclease (e.g., a selected restriction endonuclease), wherein hybridization of a complementary sequence to the single-stranded sequence generates a double-stranded recognition site that is recognized by the given nuclease. For example, hybridization of an oligonucleotide probe to a nucleic acid probe can generate a double-stranded recognition site for a nuclease, such that cleavage of the site using the nuclease releases a cleaved sequence associated with the oligonucleotide probe (and with a detectable label associated with the oligonucleotide probe). In some aspects, a double stranded sequence is cleaved. For example, the nuclease cleaves both the nucleic acid strand of the nucleic acid probe and the nucleic acid strand of the oligonucleotide probe. In this case, cleavage also releases a sequence of the nucleic acid probe (e.g., a barcode sequence to which the oligonucleotide probe hybridizes), as shown in FIG. 1 and FIG. 2A. In some embodiments, hybridization of a detection probe (or other higher-order probe, such as an L-shaped probe for formation of a branched structure) to an oligonucleotide probe can generate a double-stranded recognition site for a nuclease, such that cleavage of the site using the nuclease releases a cleaved sequence associated with the oligonucleotide probe and one or more detection probes or other higher order probes (e.g., as shown in FIG. 2B). In this case, a barcode sequence hybridized by the oligonucleotide probe can remain in the nucleic acid probe, as shown in FIG. 2B.

In some embodiments, more than one type of nucleic acid and/or oligonucleotide probes described herein may be contacted with a sample, e.g., simultaneously or sequentially in any suitable order, such as in sequential probe hybridization/unhybridization cycles. In some embodiments, more than one type of oligonucleotide probes may be contacted with a sample, e.g., simultaneously or sequentially in any suitable order, such as in sequential probe hybridization/unhybridization cycles. In some embodiments, the oligonucleotide probes hybridize to complementary hybridization regions comprised by one or more nucleic acid probes, wherein the hybridization region comprises a barcode sequence and a single stranded sequence. In some embodiments, hybridization of an oligonucleotide probe to a hybridization region comprising a single-stranded sequence generates a double-stranded recognition site that can be recognized by a nuclease (e.g., a restriction endonuclease), wherein the single-stranded sequence is not recognized by the nuclease. In some embodiments, one or more types of higher order probes may be contacted with a sample, e.g., simultaneously or sequentially in any suitable order, such as in sequential probe hybridization/unhybridization cycles. In some embodiments, more than one type of detectably labeled nucleic acid probes may be contacted with a sample, e.g., simultaneously or sequentially in any suitable order, such as in sequential probe hybridization/unhybridization cycles. In some embodiments, the detectably labeled probes may comprise probes that bind to one or more oligonucleotide probes or one or more higher order probes that are bound directly or indirectly to the oligonucleotide probes, (e.g., as in the case of a hybridization chain reaction (HCR), a branched DNA reaction (bDNA), or the like).

In some embodiments, the nucleic acid probes, oligonucleotide probes, anchor probes, and/or detection probes or other higher order probes disclosed herein may be made using only 2 or only 3 of the 4 bases, such as leaving out all the “G”s and/or leaving out all of the “C”s within the probe. Sequences lacking either “G”s or “C”s may form very little secondary structure, and can contribute to more uniform, faster hybridization in certain embodiments.

Any of the probes described herein can be linear probes. In some embodiments, a linear probe can be one that comprises a target recognition sequence and a sequence that does not hybridize to a target nucleic acid or target probe, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid or target probe but may hybridize to one another and/or one or more other probes, such as higher order probes and/or detectably labeled probes (e.g., detection probes).

In some embodiments, provided herein are probes, probe sets, and assay methods to couple target nucleic acid detection, signal amplification (e.g., through hybridization of multiple nucleic acid probes a target nucleic acid such as an RNA or an RCA product, and/or hybridization of a plurality of detectably labeled probes, such as in hybridization chain reactions and the like performed off of an overhang region of an oligonucleotide probe), and decoding of the barcode sequences. Various probes provided herein are described in further detail in Sections III A-E below.

### A. Nucleic Acid Probes

In some aspects, provided herein are nucleic acid probes for analyzing target nucleic acids in a biological sample (e.g., an analyte as described in Section II.B). In some embodiments, a nucleic acid probe provided herein comprises a target-binding region for hybridizing to a target nucleic acid in a sample. In some embodiments, a nucleic acid probe provided herein comprises a barcode region comprising one or more barcode sequences.

The target-binding region (sometimes also referred to as the targeting region/sequence or the recognition region/sequence) of a nucleic acid probe may be positioned anywhere within the probe. For instance, the target-binding sequence of a nucleic acid probe that binds to a target nucleic acid can be 5′ or 3′ to a barcode region or portion thereof in the nucleic acid probe. Likewise, the hybridization region of an oligonucleotide probe comprising a sequence complementary or substantially complementary to a barcode sequence of a nucleic acid probe can be 5′ or 3′ to an overhang region of the oligonucleotide probe, if such a region is included in the oligonucleotide probe. In some embodiments, an oligonucleotide probe herein does not comprise an overhang region. In some embodiments, an oligonucleotide probe herein does not comprise a target-binding region (e.g., for hybridizing to a sequence of the target nucleic acid). In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence of a nucleic acid probe may be determined with reference to a target nucleic acid (e.g., a cellular RNA or a reporter oligonucleotide of a labelling agent for a cellular analyte) that is present or suspected of being present in a sample. In some embodiments, more than one target-binding sequence can be used to identify a particular analyte comprising or associated with a target nucleic acid. The more than one target-binding sequence can be in the same nucleic acid probe or in different nucleic acid probes. For instance, multiple nucleic acid probes can be used, sequentially and/or simultaneously, that can bind to (e.g., hybridize to) different regions of the same target nucleic acid. In other examples, a nucleic acid probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

In some embodiments, a barcode region can be on a single overhang of a nucleic acid probe provided herein (e.g., on an overhang of an L-shaped nucleic acid probe, or on one overhang of a U-shaped probe). In other embodiments, barcode sequences of a barcode region can be positioned on two overhang regions of the nucleic acid probe. In some embodiments, a barcode region of an individual nucleic acid probe uniquely identifies a target nucleic acid. In other embodiments, a combination of the barcode regions of multiple nucleic acid probes that hybridize to the same target nucleic acid uniquely identifies the target nucleic acid. For example, a sequential signal code derived from interrogating a first barcode sequence and a second barcode sequence present on a first nucleic acid probe hybridized to the target nucleic acid and a third barcode sequence and fourth barcode sequence present on a separate nucleic acid probe hybridized to the same target nucleic acid can uniquely identify the target nucleic acid. In some embodiments, dividing the barcode sequences that identify a target nucleic acid among multiple nucleic acid probes can decrease the length requirements for the nucleic acid probes.

In some embodiments, a nucleic acid probe may comprise a barcode region, wherein the barcode region comprises 1, 2, 3, 4, 5, 6, 7, 8, 9, 10, or more barcode sequences. The barcode region may be a continuous region (e.g., a region positioned on a single overhang of a nucleic acid probe, comprising 1, 2, 3, 4, or more barcode sequences). In some embodiments, the barcode region can be split between a first and second overhang region of the nucleic acid probe.

The barcode sequences, if present, may be of any length. If more than one barcode sequence is used, the barcode sequences may independently have the same or different lengths, such as at least 5, at least 6, at least 7, at least 8, at least 9, at least 10, at least 11, at least 12, at least 13, at least 14, at least 15, at least 20, at least 25, at least 30, at least 35, at least 40, at least 50 nucleotides in length. In some embodiments, an individual barcode sequence may be no more than 30, no more than 24, no more than 16, no more than 15, no more than 14, no more than 13, no more than 12, no more than 10, no more than 9, or no more than 8 nucleotides in length. Combinations of any of these are also possible, e.g., the barcode sequence may be between 5 and 10 nucleotides, between 8 and 15 nucleotides, between 10 and 30 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes.

Nucleic acid probes and/or target nucleic acids hybridized by the nucleic acid probes can be identified using the combination of barcode sequences present in the nucleic acid probes. As an illustrative example, a first nucleic acid probe may hybridize to a first target nucleic acid, and may comprise a first barcode sequence (e.g., red), and a second barcode sequence (e.g., green), while a second, different nucleic probe may hybridize to a different target nucleic acid and comprise the same first barcode sequence (e.g., red) as in the first probe, but a different second barcode sequence instead of the second barcode sequence (e.g., blue). Such nucleic acid probes may thereby be distinguished by determining the various sequential barcode sequence combinations present or associated with a given nucleic acid probe at a given location in a sample.

In some embodiments, the barcode sequences are interspersed in the barcode region with single-stranded sequences that are single-stranded portions of a recognition site for a nuclease. The single-stranded sequence can be designed as a single-stranded portion of a double-stranded recognition site for any suitable nuclease (e.g., restriction endonuclease), wherein the nuclease does not recognize the single-stranded portion alone. In some embodiments, a plurality of barcode sequences of the barcode region can be interspersed with single-stranded portions of double-stranded recognition sites that are recognized by the same nuclease. In some embodiments, the single-stranded portions of the double-stranded recognition sites recognized by the same nuclease have the same sequence. In other embodiments, a plurality of barcode sequences of the barcode region can be interspersed with single-stranded portions of double-stranded recognition sites that are recognized by different nucleases. In some embodiments, the single-stranded portions of the double-stranded recognition sites recognized by the different nuclease have different sequences.

In some embodiments, each of the single-stranded sequences in the nucleic acid probe has the same sequence. In other embodiments, each of the single-stranded sequences in the nucleic acid probe has a different sequence. In some embodiments, at least two of the single-stranded sequences in the nucleic acid probe have different sequences. In some embodiments comprising a first, second, and third single-stranded sequence, two of the first, second, and third single-stranded sequences have the same sequence. In some embodiments comprising a first, second, and third single-stranded sequence, the first, second, and third single-stranded sequences have the same sequence. In other embodiments comprising a first, second, and third single-stranded sequence, the first, second, and third single-stranded sequences each have different sequences. In some embodiments comprising a first, second, third, and fourth single-stranded sequence, the first, second, third, and fourth single-stranded sequences have the same sequences. In some embodiments comprising a first, second, third, and fourth single-stranded sequence, two or three of the first, second, third, and fourth single-stranded sequences have the same sequences. In other embodiments comprising a first, second, third, and fourth single-stranded sequence, the first, second, third, and fourth single-stranded sequences each have different sequences.

The nuclease may recognize the double-stranded recognition site but not recognize the single-stranded sequences. In some embodiments, the single-stranded sequence is 2-6, 4-8, 2-8, 4-6, 2-10, 2-12, 2-16, 6-16, or 6-10 nucleotides in length, and the double-stranded recognition site can be 2-6, 4-8, 2-8, 4-6, 2-10, 2-12, 2-16, 6-16, or 6-10 base pairs in length. In some embodiments, the single-stranded sequence is 4, 6, or 8 nucleotides in length, and the double-stranded recognition site is 4, 6, or 8 base pairs in length. In some embodiments, the single-stranded sequence is 6 nucleotides in length, and the double-stranded recognition site is 6 base pairs in length. In some embodiments, the length of the single-stranded sequence is selected in order to minimize hybridization cross-talk (i.e., hybridization of oligonucleotide probes to non-cognate hybridization regions). In some embodiments, the GC content of the single-stranded sequence is no more than any one of 60%, 55%, 50%, 45%, 40%, 35%, 30%, 25%, or 20%. In some embodiments, the GC content of the single-stranded sequence is no more than 50%. In some embodiments, the GC content of a hybridization region (comprising a barcode sequence and a single-stranded sequence) is no more than any one of 60%, 55%, 50%, 45%, 40%, 35%, 30%, 25%, or 20%. In some embodiments, the GC of the hybridization region is no more than 50%. Nucleases capable of recognizing double-stranded recognition sites and their corresponding double-stranded recognition sites are known in the art, such as any of the restriction endonucleases available from New England Biolabs.

In some embodiments, the double-stranded recognition site is a recognition site of a restriction endonuclease. For example, in some embodiments, the double-stranded recognition site is recognized by anaI, Acc16I, AccII, AccBSI, AcvI, AfaI, AfeI, AjiI, AluBI, Aor51HI, BalI, BmgBI, Bsh1235I, BsnI, Bsp68I, BspANI, BspFNI, BsrBI, BssNAI, Bst11071, BstSNI, BsuRI, BtuMI, DinI, DraI, Ecl136II, Eco105I, Eco147I, Eco321, Eco47III, Eco53kI, Eco721, EcoICRI, EcoRV, EgeI, EheI, FspI, HpaI, HpyCH4V, KspAI, MbiI, MluNI, Mox20I, MscI, Msp20I, MssI, MvnI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, or ZrmI. In some embodiments, the double-stranded recognition site is recognized by AatII, AbsI, Acc65I, AccIII, AcII, AciI, AfIII, AgeI, AhII, Alw44I, Aor13HI, ApaI, ApaLI, AscI, AseI, AsiGI, AsiSI, Asp718I, AspA2I, AspLEI, AsuII, AvrII, BamHI, BauI, BbvCI, BcII, BcuI, BfaI, BfrI, BgIII, BlnI, BmgT120I, BmtI, Bpu14I, Bsa29I, BseAI, BseCI, BsePI, BseX3I, BseYI, BshTI, BshVI, BsiWI, BspACI, Bsp119I, Bsp120I, Bsp13I, Bsp1407I, Bsp19I, BspDI, BspEI, BspHI, BspMAI, BspOI, BspT104I, BspTI, BsrGI, BssHII, BssMI, BssSI-v2, Bst2BI, BstAFI, BstAUI, BstBI, BstHHI, BstMBI, BstZI, Bsul5I, BsuTUI CciI, CciNI, CfoI, Cfr42I, Cfr9I, ClaI, CspAI, Csp6I, CviAII, CviQI, DpnII, EagI, EclXI, EcoRI, EcoT22I, FaeI, FatI, FauNDI, FbaI, FseI, GsaI, HapII, HhaI, Hin1II, Hin6I, HindIII, HpaI, HpaII, HpySE526I, Hsp92II, KasI, Kpn2I, KpnI, Ksp22I, KspI, Kzo9I, MaeI, MboI, MluCI, MauBI, MfeI, MluI, Mly113I, Mph1103I, MreI, MroI, MroNI, MspCI, MseI, MspI, MunI, NarI, NcoI, NdeI, NdelI, NgoMIV, NheI, NlaIII, NotI, NsiI, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, RsaNI, SacI, SacII, SaII, SaqAI, Sau3AI, SbfI, SdaI, SfaAI, Sfr274I, Sfr202I, SfuI, SgfI, SgrBI, SgrDI, SgsI, SlaI, SpeI, SphI, Sse83871, Sse9I, SsiI, SspDI, SspMI, SstI, TaiI, TaqI, TaqI-v2, TasI, TrulI, TspMI, Vha464I, VneI, VspI, XbaI, XhoI, XmaI, or Zsp2I.

In some embodiments, the enzyme is capable of recognizing double-stranded recognition sites with variable sequence base pair positions. In this case, the variable positions of a single-stranded sequence for generating the double-stranded recognition site can be considered part of the barcode sequence. One example of a restriction endonuclease that recognizes double-stranded recognition sites comprising variable sequence base pair positions is BoxI, which recognizes a double-stranded sequence comprising the sequence GACNN/NNGTC, wherein “N” is any base selected from A, T, G, or C, and the cut site is indicated as “/”. In some embodiments, a variable sequence position in a double-stranded recognition site can be any one of N=A or C or G or T (any), B=C or G or T (not A), D=A or G or T (not C), H=A or C or T (not G), V=A or C or G (not T), W=A or T (weak), S=C or G (strong), R=A or G (purine), Y=C or T (pyrimidine), M=A or C (amino), or K=G or T (keto). In some aspects, the use of variable sequence positions in the single-stranded sequence can minimize hybridization cross-talk for different hybridization regions.

Examples of restriction endonucleases recognizing double-stranded recognition sites with variable sequence base positions include AasI, Acc36I, AccB7I, AcIWI, AdeI, AfiI, AhdI, Alw26I, AlwI, AlwNI, Asp700I, AspS9I, AxyI, BbsI, BccI, BcoDI, BfuAI, BgII, BlpI, Bme1390I, BmeRI, BmiI, BmrFI, Bmd, BmuI, BoxI, BpiI, Bpu10I, Bpu1102I, BsaBI, BsaJI, Bsc4I, Bse21I, Bse8I, BseDI, BseGI, BseJI, BseLI, BseMI, BsII, BsmAI, BsmBI-v2, BsmI, Bso31I, Bsp1720I, BspLI, BspMI, BspPI, BspQI, BspTNI, BsrDI, BsrI, BssECI, Bst4CI, Bst6I, BstAPI, BstC8I, BstEII, BstENI, BstF5I, BstMAI, BstMWI, BstPAI, BstPI, BstV2I, BstXI, Bsu36I, BtsCI, Btsl-v2, BtsIMutI, BveI, Cac8I, CaiI, Cfr13I, DdeI, DraIII, DrdI, DriI, DseDI, Eam1104I, Eam1105I, EarI, Eco31I, Eco81I, Eco91I, EcoNI, EcoO65I, Esp3I, FauI, Fnu4HI, Fsp4HI, HinfI, Hpyl66II, Hpy188I, Hpy188III, Hpy8I, HpyF10VI, HpyF3I, LguI, LmnI, MaeIII, MroXI, MsII, NIaIV, OliI, PaqCI, PciSI, PctI, PdmI, PflFI, PflMI, PfoI, PleI, PpsI, PshAI, PspEI, PspN4I, PspPI, PstNI, PsyI, RseI, SapI, ScrFI, SfiI, SmiMI, TaaI, Tth111I, Van91I, XagI, XcmI, and XmnI. In some embodiments, the double-stranded recognition site comprises any one of 2-6, 4-8, 2-8, 4-6, 2-10, 2-12, 2-16, 6-16, or 6-10 fixed sequence positions. In some embodiments, the double-stranded recognition site comprises at least any one of 2, 3, 4, 5, 6, 7, 8, or more fixed sequence positions. In some embodiments, the double-stranded recognition site comprises any one of 1-6, 4-8, 2-8, 4-6, 2-10, 2-12, 2-16, 1-16, or 6-10 variable sequence positions (e.g., any variable positions such as N, B, D, H, V, W, S, R, Y, M, or K, as described above).

In some embodiments, the single-stranded portion of the double-stranded recognition site is comprised by an overhang region of the oligonucleotide probe rather than the by the hybridization region of the nucleic acid probe, as shown in FIG. 2B. Thus, the barcode sequences need not be separated by the single-stranded sequences. In some aspects, this can save space on the nucleic acid probe. Additionally, in embodiments wherein the double-stranded recognition site is not generated within the nucleic acid probe, the barcode sequences of the nucleic acid probe are not cleaved, and can be arranged in any order on the nucleic acid probe.

Nucleic acid probes provided herein may additionally comprise an anchor binding region. The anchor binding region, if present, may be of any length, such as at least 5, at least 6, at least 7, at least 8, at least 9, at least 10, at least 11, at least 12, at least 13, at least 14, at least 15, at least 20, at least 25, at least 30, at least 35, at least 40, at least 50 nucleotides in length. In some embodiments, an anchor binding region may be no more than 24, no more than 16, no more than 15, no more than 14, no more than 13, no more than 12, no more than 10, no more than 9, or no more than 8 nucleotides in length. Combinations of any of these are also possible, e.g., the anchor binding region may be between 5 and 10 nucleotides, between 8 and 15 nucleotides, etc. In some embodiments, the anchor binding region is a common sequence among a plurality of nucleic acid probes.

B. Oligonucleotide probes

After contacting the nucleic acid probes with a sample, the sample can be contacted with oligonucleotide probes that hybridize to corresponding barcode sequences within the nucleic acid probes. In some embodiments, an oligonucleotide probe herein comprises (e.g., is covalently linked to) a label, or does not comprise any label. In some embodiments, an oligonucleotide probe herein comprises one or more overhang regions, wherein the overhang region(s) do not hybridize to the nucleic acid probe. The overhang region(s) may comprise detection sequences for hybridization of one or more detection probes, wherein the detection probes are associated with a label or with the absence of a label. In some embodiments, a detection sequence can comprise an initiator sequence for a signal amplification reaction, such as a hybridization chain reaction. barcode sequence in the secondary probe.

The oligonucleotide probes hybridized to the nucleic acid probes may be directly detected by determining detectable labels (if present), and/or determining the absence of a detectable label. Additionally or alternatively, the oligonucleotide probes hybridized to the nucleic acid probes can be detected by using one or more other probes that bind directly or indirectly to the probes or products thereof. The one or more other probes may comprise a detectable label.

In some embodiments, the detection may be spatiaI, e.g., in two or three dimensions. In some embodiments, the oligonucleotide probes are detected at a location in the biological sample while hybridized to the nucleic acid probe. In some embodiments, the detection may be quantitative, e.g., the amount or concentration of a nucleic acid probe (and of a corresponding target nucleic acid) may be determined. In some embodiments, the nucleic acid probes, oligonucleotide probes, and/or higher order probes (e.g., detection probes) may comprise any of a variety of entities able to hybridize a nucleic acid, e.g., DNA, RNA, LNA, and/or PNA, etc., depending on the application.

An oligonucleotide probe may contain a recognition sequence able to bind to or hybridize with a hybridization region of a nucleic acid probe, e.g., at a barcode sequence or portion(s) thereof of a barcode region in the nucleic acid probe. In some embodiments, the binding is specific, or the binding may be such that a recognition sequence preferentially binds to or hybridizes with only hybridization regions comprising one of the barcode sequences or complements thereof that are present. The oligonucleotide probe may also contain one or more detectable labels. If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In some embodiments, the length of the recognition sequence the same length as a hybridization region consisting of a barcode sequence and single-stranded sequence (i.e., a single-stranded portion of a nuclease recognition site). In some embodiments, the length of the recognition sequence the same length as a hybridization region consisting of a barcode sequence of the nucleic acid probe. In some embodiments, the recognition sequence may comprise a sequence at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, an oligonucleotide probe disclosed herein may contain or be associated with a detectable label such as a fluorophore. In some embodiments, one or more oligonucleotide probes of a plurality of oligonucleotide probes used in an assay may lack a detectable label, while one or more other oligonucleotide probes in the plurality each comprises or is associated with a detectable label selected from a limited pool of distinct detectable labels (e.g., red, green, yellow, and blue fluorophores), and the absence of detectable label may be used as a separate “color.” As such, detectable labels are not required in all cases. In some embodiments, barcode sequences in the nucleic acid probe are used to combinatorially encode a plurality of analytes of interest. As such, signals associated with the oligonucleotide probes hybridized to the nucleic acid probes at particular locations in a biological sample can be used to generate distinct sequential signal codes (sometimes referred to as signatures) that each corresponds to an analyte in the sample, thereby identifying the analytes at the particular locations, e.g., for in situ spatial analysis of the sample.

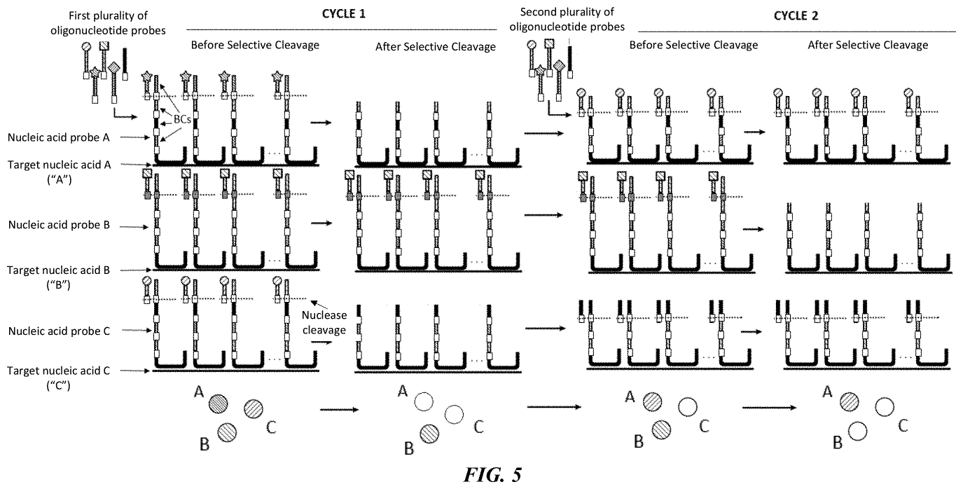

In some aspects, provided herein is a first plurality of oligonucleotide probes comprising sequences complementary to a first set of first barcode sequences. In some embodiments, provided herein is a second plurality of oligonucleotide probes comprising sequences complementary to a second set of second barcode sequences. Any number of pluralities of oligonucleotide probes corresponding to distinct sets of barcode sequences can be provided. In some embodiments, the oligonucleotide probes of one plurality of oligonucleotide probes corresponding to one set of barcode sequences are designed to avoid or minimize cross-reaction (i.e., hybridization) with barcode sequences of a different set of barcode sequences. For example, a first plurality of oligonucleotide probes may be designed such that the first oligonucleotide probes hybridize to barcode sequences of a first set of barcode sequences, but not to any subsequent barcode sequences of subsequent sets. In some aspects, this prevents or reduces premature cleavage of subsequent barcode sequences from a nucleic acid. The order of barcode sequences in a nucleic acid probe can be designed such that barcode sequences closest to one or more ends of the nucleic acid probe are detected and cleaved before barcode sequences positioned more internally, as shown in FIG. 1, FIG. 2A, FIG. 3, and FIG. 5.

C. Detection probes

In some embodiments, the oligonucleotide probes can be contacted by one or more detection probes comprising detectable labels or the absence of a detectable label, wherein the detection probes bind directly or indirectly to the oligonucleotide probe. Suitable detectable labels are described in more detail in Section V below. Exemplary probe designs and resulting hybridization complexed are shown in FIGS. 2A and 2B. Through the detection of signals associated with detection probes in a sample, the location of one or more analytes in the sample and the identity of the analyte(s) can be determined. In some embodiments, the presence/absence, absolute or relative abundance, an amount, a leveI, a concentration, an activity, and/or a relation with another analyte of a particular analyte can be analyzed in situ in the sample.

As shown in FIGS. 2A and 2B, one or more detection probes can be hybridized to the overhang of the oligonucleotide probe (e.g., L-shaped probe). In some embodiments, hybridization chain reaction (HCR) can be performed directly or indirectly on the oligonucleotide probe; linear oligonucleotide hybridization chain reaction (LO-HCR) can be performed directly or indirectly on the oligonucleotide probe; primer exchange reaction (PER) can be performed directly or indirectly on the oligonucleotide probe; assembly of branched structures can be formed directly or indirectly on the oligonucleotide probe; hybridization of a plurality of detectably labelled probes can be directly or indirectly on the oligonucleotide probe, or any combination thereof (e.g., described in Section V below).

D. Anchor probes

In some aspects, provided herein are one or more anchor probes for hybridization to an anchor binding region of one or more nucleic acid probes. A common anchor probe can be used to bind to and simultaneously detect nucleic acid probe hybridized to a plurality of target nucleic acids in the biological sample. In some embodiments, one or more anchor probes are directly or indirectly associated with a detectable label, such as any of the detectable labels described in Section V below. In some embodiments, an anchor probe comprises an overhang region (e.g., a 5′ and/or 3′ overhang region) for binding to one or more detection probes and/or higher order probes (e.g., for any of the signal amplification strategies described herein, such as hybridization of a plurality of detectably labeled detection probes to an overhang of the anchor, or hybridization chain reactions, primer exchange reactions, or formation of branched hybridization structures off of an overhang region of the anchor probe. Suitable signal amplification strategies are described in Section V below.

E. Additional probe types

In some aspects, provided herein are one or more types of additional probes, such as circular or circularizable probes and probe sets (e.g., for performing rolling circle amplification). and/or one or more higher order probes (e.g., for hybridization to an overhang region of an oligonucleotide probe to provide signal amplification).

In some embodiments, a target nucleic acid is an RCA product. Any circular or circularizable probe or probe set can be used to generate the RCA product that is the target nucleic acid for a nucleic acid probe described herein. Suitable circular or circularizable probes or probe sets are described in Section II (iii), related to products of endogenous analytes and/or labelling agents.

In some aspects, provided herein are one or more additional higher order probes for hybridization to an oligonucleotide probe, e.g., for signal amplification. Suitable higher order probes can include L-shaped probes (e.g., one that comprises a hybridization region that binds to the oligonucleotide probe and a 5′ or 3′ overhang upon hybridization to the oligonucleotide probe), or a U-shaped probe (e.g., one that comprises a hybridization region that binds to the oligonucleotide probe, a 5′ overhang, and a 3′ overhang upon hybridization to the oligonucleotide probe). Higher order probes can also include probes for hybridization chain reactions or primer exchange reactions, such as any of the probes described in Section V below.

### IV. Signal Detection and Decoding

A. Decoding Using Sequential Oligonucleotide Probe Hybridization

In some aspects, provided herein is a method for decoding a barcode region of one or more nucleic acid probes by sequential hybridization and detection of oligonucleotide probes to barcode sequences of the barcode region. The detection of labels or the absence of labels associated with the sequentially hybridized oligonucleotide probes generates a sequential signal code (sometimes referred to as a signature or a codeword). In some embodiments, the sequential signal code identifies a target nucleic acid (e.g., a target analyte or target nucleic acid associated with a target analyte) in the sample.

Sequential signal codes for each target may be assigned sequentially, or may be assigned at random. For instance, referring to FIG. 3, a target nucleic acid A may be assigned to a sequential signal code A, while a target nucleic acid B may be assigned to sequential signal code B. In addition, in some embodiments, the sequential signal codes may be assigned using an error-detection system or an error-correcting system, such as a Hamming system, a Golay code, or an extended Hamming system (or a SECDED system, i.e., single error correction, double error detection). Generally speaking, such systems can be used to identify where errors have occurred, and in some cases, such systems can also be used to correct the errors and determine what the correct sequential signal code should have been. For example, a sequential signal code such as 001 may be detected as invalid and corrected using such a system to 101, e.g., if 001 is not previously assigned to a different target sequence. It should also be understood that all possible sequential signal codes of a given encoding scheme need not be used in some cases. For example, in some embodiments, sequential signal codes that are not used can serve as negative controls. Similarly, in some embodiments, some sequential signal codes can be left out because they are more prone to errors in measurement than other sequential signal codes.

In some embodiments, a method disclosed herein comprises sequential hybridization of oligonucleotide probes labeled (directly or indirectly) with one color (e.g., the same fluorophore or fluorophores with the same emission wavelength), or lacking a label. In some instances, a method disclosed herein comprises sequential hybridization of oligonucleotide probes labeled with one color to a nucleic acid probe (e.g., as shown in FIGS. 3 and 5), where the nucleic acid probe is hybridized to a target nucleic acid. The target nucleic acid can be any of the target nucleic acids described in Section II, such as an endogenous nucleic acid analyte (e.g., mRNA) or an amplification product (e.g., an RCA product, such as an RCA product of a circular or circularized probe). In some embodiments, the nucleic acid probe is hybridized to an amplification product (e.g., an RCA product) of a primary probe (e.g., a padlock probe or a SNAIL probe pair), wherein the primary probe hybridizes to a target nucleic acid, such as an mRNA molecule.

An exemplary method comprises contacting the sample with a plurality of oligonucleotide probes, wherein each oligonucleotide probe of the plurality is associated with a distinct label or the absence of a label. Each oligonucleotide probe of the plurality can comprise a sequence complementary to a barcode sequence of a set of barcode sequences. Thus, by assigning a distinct detectable label or the absence of a label to each barcode sequence of the set of barcode sequences, a barcode sequence can be determined at a spatially resolved location in the sample by detecting the label or absence thereof of the oligonucleotide probe hybridized at that location. The identity of the barcode region can be determined based on the sequential determination of barcode sequences of the barcode region. In some embodiments, the barcode region uniquely corresponds to the nucleic acid probe (i.e., to a target sequence hybridized by the nucleic acid probe). In some embodiments, the barcode region uniquely identifies a target nucleic acid bound by the nucleic acid probe. For example, the same barcode region can be present in a group of nucleic acid probes that hybridize to different target sequences comprised by the same target nucleic acid.

In the example shown in FIG. 3, each nucleic acid probe comprises a barcode region comprising a first barcode sequence, a second barcode sequence, a third barcode sequence, and a fourth barcode sequence. The first barcode sequence can be selected from a set of first barcode sequences, each assigned to a different label or to the absence of a label. In FIG. 3, a first set of barcode sequences (BCs) comprises 4 first barcode sequences, wherein the 4 first barcode sequences are assigned to one of four distinct signals (e.g., one of four distinct detectable labels, or one of four distinct signals wherein three of the distinct signals are distinct detectable labels and one of the distinct signals is the absence of a label). Similarly, the second barcode sequence can be selected from a set of second barcode sequences, each assigned to a different label or to the absence of a label. The labels used in the first set and the second set can be the same labels, because the oligonucleotide probe sequence associated with the label can be cleaved using a nuclease between detection cycles. The third barcode sequence can similarly be selected from a set of third barcode sequences, each assigned to a different label or to the absence of a label, and the fourth barcode sequence can be selected from a set of fourth barcode sequences, each assigned to a different label or to the absence of a label.

The coding capacity of the encoding and decoding scheme depicted in FIG. 3 may depend on the number of sequential barcode sequences comprised by the nucleic acid probe, and the number of different oligonucleotide probes for each barcode sequence. For example, if nucleic acid probe comprises four barcode sequences that are sequentially detected by hybridization of oligonucleotide probes, and the number of distinct barcode sequences (corresponding to distinguishable labels or the absence of a label) is four, then the coding capacity of the method can be 4⁴, or 256. In some embodiments, the oligonucleotide probes corresponding to each of the first, second, third, and fourth barcode sequence do not cross react with any barcode sequences of any other set of barcode sequences. For example, the oligonucleotide probes designed to hybridize to the first barcode sequences of the first set of barcode sequences may not hybridize to any of the second set of second barcode sequences, the third set of barcode sequences, or the fourth set of barcode sequences. Thus, in the example above using four sets of barcode sequences and four distinct oligonucleotide probes for determination of each barcode sequence, the number of oligonucleotide probes required to decode up to 256 different barcode regions is 16.

In the example shown in FIG. 5, oligonucleotide probes that hybridize to the nucleic acid probes can be selectively cleaved during each cycle due to unique double-stranded recognition site sequences generated by hybridization of the oligonucleotide probe to the single-stranded sequence in the nucleic acid probe, which can then each be recognized by a unique nuclease. This allow for expanding the number of imaging rounds where a sequential signal code corresponding to each target nucleic acid is generated. In some aspects, the selective removal allows certain signals to be detected across two or more cycles. In some cases, this may allow more combinations of signals in the sequential signal codes used to identify a plurality of target nucleic acids and achieve higher plexy in the methods of assaying a plurality of analytes.

In some embodiments, to distinguish different genes (e.g., mRNA transcripts from different genes), each gene is barcoded by hybridization of one or more nucleic acid probes (e.g., 20-60 nucleic acid probes), wherein a nucleic acid probe comprises a barcode region comprising a plurality of barcode sequences. The barcode sequences can be detected by sequential rounds of hybridization of oligonucleotide probes to the nucleic acid probe.

In some aspects, unlike seqFISH and seqFISH+, the systems and methods disclosed herein do not require the design of a large set of target-specific probes that all hybridize to the same target nucleic acid molecule but to different sequences (e.g., 24-32 primary probes that hybridize to the same mRNA or cDNA molecule). For example, using a method disclosed herein, because the signal associated with the oligonucleotide probe can be amplified (e.g., an oligonucleotide probe can be targeted by multiple molecules of the same detection oligonucleotide, resulting in signal amplification), there is no need to design more than one, more than two, more than three, more than four, more than five, more than six, more than seven, more than eight, more than nine, or more than 10 primary probes that hybridize to different sequences of the same target nucleic acid molecule in order to achieve a detectable signal intensity.

In some embodiments, after hybridization of a fluorescently labeled oligonucleotide that detects a barcode sequence (e.g., on a nucleic acid probe), and optionally washing away the unbound molecules of the oligonucleotide probe, the sample is imaged and a sequence associated with the oligonucleotide or detectable label is removed by nuclease cleavage of a double-stranded recognition site generated by hybridization of the oligonucleotide probe. Then the sample is re-hybridized in a subsequent round with a fluorescently labeled oligonucleotide that detects another barcode sequence, and the oligonucleotide can be labeled with the same color or a different color as the fluorescently labeled oligonucleotide of the previous cycle. In some embodiments, as the positions of the analytes, probes, and/or products thereof can be fixed (e.g., via fixing and/or crosslinking) in a sample, the fluorescent spot corresponding to an analyte, probe, or product thereof remains in place during multiple rounds of hybridization and can be aligned to read out a string of signals in the form of a sequential signal code. Each analyte (e.g., mRNA species) can therefore be assigned a unique sequential signal code.

Barcode sequence allocation on nucleic acid probes can be in any suitable combination. In some embodiments, to visualize the different transcripts, the nucleic acid probes contain barcode regions unique to each gene. For example, each nucleic acid probe may contain four or six barcode sequences, and the 4-unit or 6-unit barcode sequence combination is unique to the mRNA transcript that the nucleic acid probe hybridizes to. In some embodiments, barcode sequences allocated among different nucleic acid probes that hybridize to the same target nucleic acid can provide a barcode sequence combination that is unique to the target nucleic acid (e.g., the mRNA transcript) that the nucleic acid probes hybridize to. For example, there can be two barcode sequences in one nucleic acid probe and four barcode sequences in another nucleic acid probe, and the combination of the six barcode sequences can be unique to the target nucleic acid (e.g., an endogenous target nucleic acid or a probe or product thereof), thus the analyte of interest can be assigned a unique sequential signal code corresponding to a sequential pattern of oligonucleotide probes associated with detectable labels or the absence of a label binding to the barcode sequences in the nucleic acid probes. Hybridization with fluorophore labeled oligonucleotide probes or detection probes allows the readout of these barcode sequences and fluorescently labels the subset of target nucleic acids (e.g., the subset of mRNAs) that contain the corresponding sequences.

In some embodiments, target nucleic acids (e.g., mRNAs) in a tissue sample may be sampled every 2, 3, 4, 5, 6, 7, 8, 9, 10, 11, 12, 13, 14, 15, 16, 17, 18, 19, 20, or more than 20 rounds of readout hybridization and collapsed into resolved images. In some embodiments, the nucleic acid probe contains six barcode sequences, and the genes are sampled every three sequential hybridization and imaging rounds. The oligonucleotide probes can be associated with distinct labels (e.g., distinct fluorophores) or the absence of a label. Thus, a total of 6×3=18 sequential hybridization/imaging rounds may be performed. Thus, a total of 18 rounds of hybridizations can enumerate the 6-unit barcode for each target nucleic acid, with the target nucleic acids in a tissue sample sampled every 3 rounds of readout hybridization and collapsed into resolved images. With 6 barcodes per target and 3 rounds of hybridization, 6³=216 unique nucleic acid targets can be profiled.

In some embodiments, a probe disclosed herein (e.g., a nucleic acid probe, oligonucleotide probe, and/or higher order probe) is capable of giving rise to signal by being detected, either directly or indirectly. In any given cycle, the presence or absence of signal may be informative and can be included as part of a sequential signal code or signature. Thus, one or more dark cycles can be incorporated into an encoding/decoding scheme to supplement detection channels corresponding to the detectable labels used in the detectably labeled probes. In other words, the absence of a detectable signal may be used as a “color,” e.g., in addition to the limited number of available fluorescent color channels in fluorescent microscopy, dark cycles can be used to alleviate issues associated with optical crowding in one or more color channels. In some aspects, a dark cycle can be incorporated by use of an unlabeled oligonucleotide probe to hybridize to the nucleic acid probe. In some aspects, a dark cycle can be incorporated by not providing or hybridizing any oligonucleotide probe to the nucleic acid probe for a given cycle. Different probes may be detected, or distinguished from one another, by different labels, or by absence of a detectable label. In some embodiments, a probe may be directly or indirectly labelled with a detectable label which gives rise to a signal which may be recorded and/or assigned (e.g., serially) a signal code. In some embodiments, a probe is capable of hybridizing to a different target nucleic acid sequence (e.g., barcode sequence corresponding to a target analyte) and providing a signal. In some embodiments, a signal may include the signal detectable from the detectable label, and different detectable labels may provide different signals which may be distinguished, e.g. by color. In some embodiments, absence of signal may also be recorded and/or assigned a signal code. In some embodiments, in a plurality of probes, one or more of the probes may be lacking a detectable label, and thus the absence of a signal may be recorded and analyzed, for example, by assigning a signal code to the absence of signal (also known as a “dark” cycle for the one or more of the probes and the corresponding analyte(s)). In some embodiments, when there is a single cycle of detection to detect the signals from the oligonucleotide probes, the plurality of oligonucleotide probes may comprise molecules of one oligonucleotide probe which is not labelled, and the remainder of the oligonucleotide probes may comprise detectable labels which can be distinguished from one another. In some embodiments, a combinatorial, e.g. sequential, labelling scheme is used (e.g., multiple cycles of sequential signal detection), and the plurality of oligonucleotide probes for different nucleic acid probes used in a given cycle need not all be distinguishable from one another in terms of the signal (e.g., may comprise the same detectable label, such as the same color of fluorophore), as it is the combination (e.g., sequence or order) of signals which identifies the target nucleic acid sequence, not a single signal.

The detectable label may be any detectable moiety and may be directly or indirectly linked to the oligonucleotide probe. The oligonucleotide probe may thus be considered to be directly or indirectly signal-providing. In some embodiments, the detectable label is incorporated into the oligonucleotide probe. For example, the detectable label may be linked directly (e.g., covalently) or via a linker (e.g., a chemical or nucleic acid linker) to the hybridization region of the oligonucleotide probe that binds to a complementary hybridization region of a nucleic acid probe.

### B. Generation and Cleavage of Double-Stranded Recognition Sites

In some aspects, the methods provided herein comprise generation of a double-stranded recognition site that is recognized by a nuclease, wherein the double-stranded recognition site is generated by hybridization of a probe to a single-stranded hybridization region of another probe. In some embodiments, the double-stranded recognition sequence is generated by hybridization of an oligonucleotide probe to a hybridization region of a nucleic acid probe, wherein the hybridization region comprises a barcode region and a single-stranded sequence that is a single-stranded portion of the double-stranded recognition sequence (e.g., as shown in FIG. 1 and FIG. 2A). In some embodiments, the double-stranded recognition site is generated by hybridization of a detection probe or other higher order probe to an oligonucleotide probe, wherein the oligonucleotide probe is hybridized to a barcode sequence of a nucleic acid probe (e.g., as shown in FIG. 2B). The single-stranded sequence is not recognized by the nuclease.

In some embodiments, the method comprises contacting the biological sample with a nuclease, such as a restriction endonuclease. In some embodiments, the sample is contacted with at least any of 1 U, 2 U, 5 U, 10 U, 20 U, 30 U, 40 U, 50 U, 60 U, 70 U, 80 U, 90 U, or 100 U of a restriction endonuclease. One unit of restriction endonuclease activity is defined as the amount of enzyme (measured in units, U) that will cleave 1 μg of DNA (usually lambda DNA) to completion in 1 hour at the optimum temperature for the enzyme, usually 37° C. In some embodiments, the endonuclease is a ribonuclease.

Suitable restriction endonucleases include restriction endonucleases that generate blunt ends or sticky ends. Exemplary restriction endonucleases include anaI, Acc16I, AccII, AccBSI, AcvI, AfaI, AfeI, AjiI, AluBI, Aor51HI, BalI, BmgBI, Bsh1235I, BsnI, Bsp68I, BspANI, BspFNI, BsrBI, BssNAI, Bst11071, BstSNI, BsuRI, BtuMI, DinI, DraI, Ecl136II, Eco105I, Eco1471, Eco321, Eco47III, Eco53kI, Eco721, EcoICRI, EcoRV, EgeI, EheI, FspI, HpaI, HpyCH4V, KspAI, MbiI, MluNI, Mox20I, MscI, Msp20I, MssI, MvnI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, ZrmI, AatII, AbsI, Acc65I, AccIII, AcII, AciI, AfIII, AgeI, AhII, Alw44I, Aor13HI, ApaI, ApaLI, AscI, AseI, AsiGI, AsiSI, Asp718I, AspA2I, AspLEI, AsulI, AvrlI, BamHI, BauI, BbvCI, BcII, BcuI, BfaI, BfrI, BgIII, BlnI, BmgT120I, BmtI, Bpu14I, Bsa29I, BseAI, BseCI, BsePI, BseX3I, BseYI, BshTI, BshVI, BsiWI, BspACI, Bsp119I, Bsp120I, Bsp13I, Bsp1407I, Bsp19I, BspDI, BspEI, BspHI, BspMAI, BspOI, BspT104I, BspTI, BsrGI, BssHII, BssMI, BssSI-v2, Bst2BI, BstAFI, BstAUI, BstBI, BstHHI, BstMBI, BstZI, Bsu15I, BsuTUI CciI, CciNI, CfoI, Cfr42I, Cfr9I, ClaI, CspAI, Csp6I, CviAII, CviQI, DpnII, EagI, EclXI, EcoRI, EcoT22I, FaeI, FatI, FauNDI, FbaI, FseI, GsaI, HapII, HhaI, HinlII, Hin6I, HindIII, HpaI, HpaII, HpySE526I, Hsp92II, KasI, Kpn2I, KpnI, Ksp22I, KspI, Kzo9I, MaeI, MboI, MluCI, MauBI, MfeI, MluI, Mlyl13I, Mph1103I, MreI, MroI, MroNI, MspCI, MseI, MspI, MunI, NarI, NcoI, NdeI, NdelI, NgoMIV, NheI, NlaIII, NotI, NsiI, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, RsaNI, SacI, SacII, SaII, SaqAI, Sau3AI, SbfI, SdaI, SfaAI, Sfr274I, Sfr202I, SfuI, SgfI, SgrBI, SgrDI, SgsI, SlaI, SpeI, SphI, Sse83871, Sse9I, SsiI, SspDI, SspMI, SstI, TaiI, TaqI, TaqI-v2, TasI, TrulI, TspMI, Vha464I, VneI, VspI, XbaI, XhoI, XmaI, or Zsp2I. In some embodiments, the restriction endonuclease is EcoRV.

In some embodiments, the restriction endonuclease is capable of recognizing double-stranded recognition sites with variable or degenerate sequence base positions. Examples of restriction endonucleases recognizing double-stranded recognition sites with variable sequence base positions include AasI, Acc36I, AccB7I, AcIWI, AdeI, AfiI, AhdI, Alw26I, AlwI, AlwNI, Asp700I, AspS9I, AxyI, BbsI, BccI, BcoDI, BfuAI, BgII, BlpI, Bme1390I, BmeRI, BmiI, BmrFI, BmrI, BmuI, BoxI, BpiI, Bpu10I, Bpu1102I, BsaBI, BsaJI, Bsc4I, Bse21I, Bse8I, BseDI, BseGI, BselI, BseLI, BseMI, BsII, BsmAI, BsmBI-v2, BsmI, Bso31I, Bsp1720I, BspLI, BspMI, BspPI, BspQI, BspTNI, BsrDI, BsrI, BssECI, Bst4CI, Bst6I, BstAPI, BstC8I, BstEII, BstENI, BstF5I, BstMAI, BstMWI, BstPAI, BstPI, BstV2I, BstXI, Bsu36I, BtsCI, Btsl-v2, BtsIMutI, BveI, Cac8I, CaiI, Cfr13I, DdeI, DraIII, DrdI, DriI, DseDI, Eam1104I, Eam1105I, EarI, Eco31I, Eco81I, Eco91I, EcoNI, EcoO65I, Esp3I, FauI, Fnu4HI, Fsp4HI, HinfI, Hpy166II, Hpy188I, Hpy188III, Hpy8I, HpyF10VI, HpyF3I, LguI, LmnI, MaeIII, MroXI, MsII, NIaIV, OliI, PaqCI, PciSI, PctI, PdmI, PflFI, PflMI, PfoI, PleI, PpsI, PshAI, PspEI, PspN4I, PspPI, PstNI, PsyI, RseI, SapI, ScrFI, SfiI, SmiMI, TaaI, Tth111I, Van91I, XagI, XcmI, and XmnI.

In some embodiments, the method comprises hybridizing a plurality of first oligonucleotide probes to complementary first hybridization regions of nucleic acid probes in the sample; wherein the first hybridization region comprises a first barcode sequence of the barcode region and a first single-stranded sequence, wherein hybridization of the oligonucleotide probe to the first single-stranded sequence creates a double stranded recognition site that is recognized by a nuclease; c) detecting a signal or absence thereof associated with the oligonucleotide probe; and d) cleaving the double stranded recognition site using the nuclease, thereby releasing a cleaved sequence associated with the oligonucleotide probe. In some embodiments, cleaving the double-stranded recognition site comprises incubating the sample with the nuclease (e.g., a restriction endonuclease). In some embodiments, the sample is incubated with the nuclease (e.g., a restriction endonuclease) after detecting a signal or absence thereof associated with the oligonucleotide probe. In some embodiments, after imaging the sample and before contacting the sample with a subsequent plurality of oligonucleotide probes, the method comprises incubating the sample with the nuclease (e.g., a restriction endonuclease) for at least 20 minutes, at least 30 minutes, at least 35 minutes, at least 40 minutes, at least 45 minutes, at least 50 minutes, at least 60 minutes, at least 80 minutes, at least 100 minutes, or at least 120 minutes. In some embodiments, the incubation with the nuclease is performed for 20-60 minutes, 20-45 minutes, 20-120 minutes, 30-120 minutes, 30-60 minutes, or 30-90 minutes. In some embodiments, the incubation with the restriction endonuclease is performed at 30° C-40° C., e.g., at 37° C.

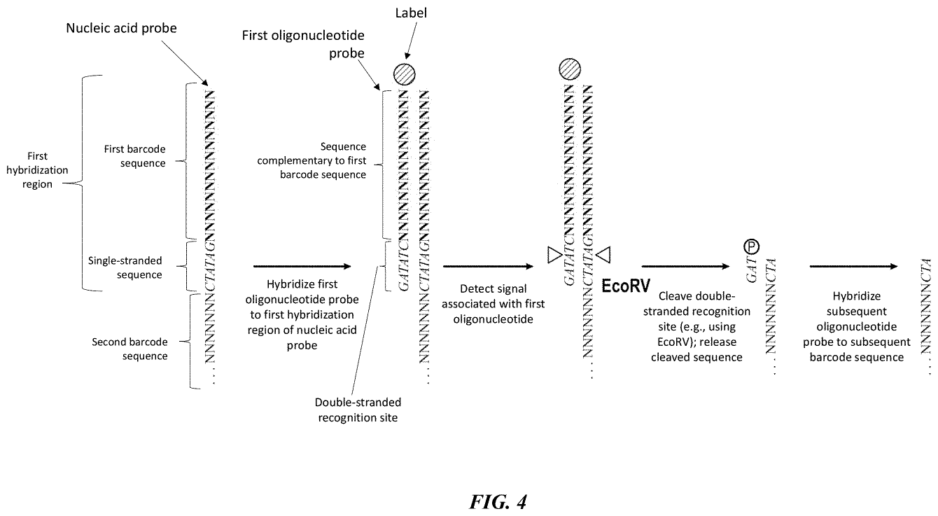

In some embodiments, the biological sample can be contacted with a nuclease (e.g., a restriction endonuclease) prior to each cleavage step. In some embodiments, a cleavage step is followed by a wash step to remove a cleaved sequence associated with the oligonucleotide probe from the sample. In some aspects, the cleaved sequence is not detected or analyzed. In some embodiments, the wash step additionally removes a second portion of the oligonucleotide probe generated by cleavage of the double-stranded recognition site (e.g., a 2, 3, or 4 nucleotide fragment of the oligonucleotide probe, as shown in FIG. 4. In some embodiments, the melting temperature of the second portion of the oligonucleotide probe for the nucleic acid probe is lower than the melting temperature of the nucleic acid probe for the target nucleic acid, such that the remaining fragment is washed away without removing the nucleic acid probe from the target nucleic acid. In some embodiments. the melting temperature (Tₘ) of the remaining fragment of the oligonucleotide probe is less than 20 ° C., less than 22 ° C., less than 25 ° C., less than 30 ° C., or less than 35 ° C. In some embodiments, the removal step comprises performing a stringent wash.

In some embodiments, a single nuclease is used to cleave all of the double-stranded recognition sites generated in the various cycles of the decoding method. In some aspects, the ability of the nuclease to recognize the double-stranded recognition site but not the single-stranded sequence enables the same sequence to be reused between multiple barcode sequences of the nucleic acid probe without premature or otherwise undesirable cleavage of barcode sequences.

In some embodiments, such as shown in FIG. 5, a single nuclease is used to selectively cleave one or more of the double-stranded recognition sites generated in the various cycles of the decoding method. For example, the sample as shown in FIG. 5 is incubated with a restriction endonuclease that recognizes the double-stranded recognition sites generated by hybridization of the first oligonucleotide probes to the nucleic acid probes on target nucleic acid A and target nucleic acid C, thereby resulting in their cleavage, whereas the double-stranded recognition site generated by hybridization of the first oligonucleotide probes to the nucleic acid probes on target nucleic acid B is not recognized and thus not cleaved by the restriction endonuclease that is incubated with the sample during cycle 1. Similar selective cleavage can also occur in subsequent cycles, such as in cycle 2 as shown in FIG. 5. In some embodiments, a single nuclease does not cleave all of the double-stranded recognition sites generated in the various cycles of the decoding method. In some embodiments, the double-stranded recognition sites generated in the various cycles of the decoding method are cleaved using two or more different nucleases that each cleave a different double-stranded recognition site. In some aspects, the ability of a nuclease to recognize some, but not all, of the double-stranded recognition sites generated in a cycle allows for the selective cleavage, and thus removal, of some of the oligonucleotide probes that were added in that cycle, thereby expanding the number of rounds of imaging and sequential signal code generation that occurs during each cycle. In some embodiments, a plurality of oligonucleotide probes that are not labelled is selectively not cleaved in one or more cycles of the decoding method to incorporate one or more dark cycles in the decoding scheme. In some embodiments, upon hybridization of an oligonucleotide probe to a barcode region of a nucleic acid probe, the nucleic acid probe and the oligonucleotide probe hybridized thereon do not form a three-way junction with another nucleic acid molecule. In some embodiments, the oligonucleotide probe may hybridize to another nucleic acid molecule; while the duplex formed between the oligonucleotide probe and the nucleic acid probe can be cleaved by a nuclease, the duplex formed between the oligonucleotide probe and the another nucleic acid molecule may, but in some examples does not, comprise a nuclease recognition site or cleavage site. In some embodiments, detecting a signal (or absence thereof) associated with the oligonucleotide probe does not depend on cleavage by a nuclease or the detection of a cleavage product.

In some embodiments, the nuclease (e.g., a restriction endonuclease) is a rare cutter for endogenous sequences in the biological sample. Thus, the digestion of endogenous sequences such as chromosomal DNA can be minimized by selection of the restriction endonuclease.

In some embodiments, a single nuclease (e.g., a single restriction endonuclease) is used to cleave the double-stranded recognition sites for the plurality of oligonucleotide probes hybridized in the sample. In other embodiments, two or more restriction endonucleases can be used to cleave different double-stranded recognition sites in the sample. In some embodiments, two or more restriction endonucleases can be combined in the same incubation to cleave different double-stranded recognition sites of the same cycle. In some embodiments, the same one or more restriction endonucleases is contacted with the sample repeatedly to cleave double-stranded recognition sites in multiple cycles of decoding and cleavage. In some embodiments, different restriction endonucleases can be used in different cycles. In some aspects, the use of different restriction endonucleases in different cycles (and different corresponding single-stranded sequences) facilitates the design of oligonucleotide probes that do not cross react with hybridization regions of different cycles. In some embodiments, some of the double-stranded recognition sites formed are not cleaved in a particular cycle while others are cleaved. In an exemplary workflow such as that depicted in FIG. 3, a method disclosed herein can comprise contacting a biological sample with a plurality of nucleic acid probes, wherein each nucleic acid probe comprises a barcode region comprising one or more barcode sequences, and wherein each nucleic acid probe hybridizes to a cognate target nucleic acid in the biological sample (e.g., nucleic acid probes A hybridize to target nucleic acid A, nucleic acid probes B hybridize to target nucleic acid B, nucleic acid probes C hybridize to target nucleic acid C, and so on). In some embodiments, the method comprises contacting the sample with a first set of first oligonucleotide probes that hybridize to corresponding first hybridization regions of the nucleic acid probes in the sample, wherein the first hybridization regions comprise first barcode sequences and single-stranded sequences. Hybridization of a first oligonucleotide probe to a given first hybridization region can be used to identify the first barcode sequence (e.g., by detecting a signal or absence thereof associated with the first oligonucleotide probe hybridized to the first barcode region), and can generate a double-stranded recognition site that is recognized by a nuclease. After detecting the signal or absence thereof associated with the first oligonucleotide probe, the double-stranded recognition sites can be cleaved using the nuclease, thereby releasing a cleaved sequence associated with the first oligonucleotide probe. Cycles of hybridizing, detecting, and cleaving can be repeated with a second set of oligonucleotide probes, a third set of nucleotide probes, and a fourth set of nucleotide probes, until all barcode sequences of the barcode regions of the nucleic acid probes have been decoded.

Although FIG. 3 depicts an example wherein the barcode region comprises four barcode sequences, any number of barcode sequences may be used to identify the desired number of target nucleic acids. In some embodiments, the last hybridization region (e.g., the fourth hybridization region depicted in FIG. 3) comprises a single-stranded sequence, and hybridization of the final oligonucleotide probe (e.g., the fourth oligonucleotide probe in FIG. 3) generates a double-stranded recognition site that is recognized by a nuclease, as shown in FIG. 3. In some embodiments, cleavage to release a sequence of the final oligonucleotide probe is followed by hybridization of an anchor probe to simultaneously detect a plurality of target nucleic acids in the sample. In other embodiments, the final hybridization region does not comprise a single-stranded sequence for generating a double-stranded recognition site, and/or the method does not comprise cleaving a double-stranded recognition site to release a sequence associated with the final oligonucleotide probe. In some embodiments, selective cleavage of certain oligonucleotide probes based on their unique double-stranded recognition sites is performed, such as shown in FIG. 5.

In some aspects wherein an oligonucleotide probe lacking a label is used for a particular barcode sequence, the oligonucleotide probe and/or barcode sequence need not be removed by nuclease cleavage. Thus, in some aspects, a single-stranded portion of a double-stranded recognition site is omitted from a hybridization region comprising a barcode sequence corresponding to an oligonucleotide probe lacking a label. In any of the embodiments wherein a barcode sequence corresponds to “dark” or the absence of a label, an oligonucleotide probe complementary to the barcode sequence may be omitted from the plurality of oligonucleotide probes rather than including an oligonucleotide probe. In this case, the single-stranded sequence of the hybridization region comprising the barcode sequence is not converted to a double-stranded recognition site and is not cleaved by the nuclease.

### V. Signal Amplification, Detection and Analysis

In some aspects, the provided methods involve analyzing, e.g., detecting or determining, one or more barcode sequences of a barcode region in a nucleic acid probe, wherein the combination of barcode sequences of the barcode region defines a sequential signal code. The sequential signal code can identify a target nucleic acid or a target sequence thereof that is hybridized by the nucleic acid probe. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative controI, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

In some embodiments, a method disclosed herein may also comprise one or more signal amplification components. In some embodiments, the present disclosure relates to the detection of nucleic acid sequences in situ using probe hybridization and generation of amplified signals associated with the probes, wherein background signal is reduced and sensitivity is increased. In some embodiments, the target nucleic acid of a nucleic acid probe comprises multiple target sequences for nucleic acid probe hybridization, such that the signal corresponding to a barcode sequence of the nucleic acid probe is amplified by the presence of multiple nucleic acid probes hybridized to the target nucleic acid. For example, multiple sequences can be selected from a target nucleic acid such as an mRNA, such that a group of nucleic acid probes (e.g., 20-50 nucleic acid probes) hybridize to the mRNA in a tiled fashion. In another example, the target nucleic acid can be an amplification product (e.g., an RCA product) comprising multiple copies of a target sequence (e.g., a barcode sequence of the RCA product).

Alternatively or additionally, amplification of a signal associated with a barcode sequence of a nucleic acid probe can be amplified using one or more signal amplification strategies off of an oligonucleotide probe that hybridizes to the barcode sequence. In some aspects, amplification of the signal associated with the oligonucleotide probe can reduce the number of nucleic acid probes needed to hybridize to the target nucleic acid to obtain a sufficient signal-to-noise ratio. For example, the number of nucleic acid probes to tile a target nucleic acid such as an mRNA can be reduced. In some aspects, reducing the number of nucleic acid probes tiling a target nucleic acid enables detection of shorter target nucleic acids, such as shorter mRNAs. In some embodiments, no more than one, two, three, 4, 5, 6, 7, 8, 9, 10, 11, 12, 13, 14, 15, 16, 17, 18, 19, or 20 nucleic acid probes may be hybridized to the target nucleic acid. In embodiments wherein the target nucleic acid is an amplification product, signal amplification off of the oligonucleotide probes may reduce the number of target sequences required for detection (e.g., the length of the RCA product can be reduced).

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, WO 2020/102094, WO 2020/163397, and WO 2021/067475, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al., 2008 Doklady Biochemistry and Biophysics, 419, 53-55; Niu et al., 2010, 46, 3089-3091; Choi et al., 2010, Nat. Biotechnol. 28(11), 1208-1212; and Song et al., 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 incorporated herein by reference), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes combination of the sequential decoding methods described herein with an assembly for branched signal amplification. In some embodiments, the assembly complex comprises an amplifier hybridized directly or indirectly (via one or more oligonucleotides) to a sequence of an oligonucleotide probe described herein. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, an oligonucleotide probe described herein can be associated with an amplified signal by a method that comprises signal amplification by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer (see e.g., U.S. Pat. Pub. No. US20190106733, which is incorporated herein by reference, for exemplary molecules and PER reaction components).

In some aspects, the provided methods comprise imaging the biological sample comprising the target nucleic acids and detecting labels of the absence thereof associated with oligonucleotide probes hybridized to barcode sequences of nucleic acid probes in the sample. In some embodiments, the oligonucleotide probes are associated with labels by the hybridization of detection probes. In some embodiments, the detection probe comprises a detectable label that can be measured and quantitated. The terms “label” and “detectable label” comprise a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyI, umbelliferone, Texas red, luminoI, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, an oligonucleotide probe or detection probe containing a detectable label can be used to detect one or target nucleic acids described herein. In some embodiments, the methods involve incubating the oligonucleotide probe with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacteriaI, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR-™488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488 UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, DansyI, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In generaI, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyI, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a- digoxigenin, dinitrophenol (DNP)/a-DNP, 5-Carboxyfluorescein (FAM)/a-FAM.

In some embodiments, a nucleotide and/or an polynucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenoI, dansyI, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the analysis of the barcode region by imaging hybridized oligonucleotide probes can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signaI, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the oligonucleotide probe or detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the oligonucleotide probe or detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low- voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the barcodes of the probes (e.g., the nucleic acid probes) are targeted by oligonucleotide probes associated with detectable labels or with the absence of labels, such as fluorescently labeled oligonucleotide probes. In some embodiments, one or more decoding schemes are used to decode the signals, such as fluorescence, for sequence determination. Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

### VI. Kits

In some aspects, provided herein are kits for analyzing an analyte in a biological sample according to any of the methods described herein. In some embodiments, provided herein is a kit comprising one or more of the probes disclosed herein, including the nucleic acid probes and/or oligonucleotide probes described in Section III. In some embodiments, the kit comprises anchor probes. In some embodiments, a set of nucleic acid probes are designed and provided for each target and the kit may comprise a plurality of sets of nucleic acid probes for a plurality of targets.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents for performing a nuclease digest described herein, such as one or more restriction endonuclease enzymes and buffers for restriction digest reactions. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

In some aspects, provided herein is a kit for analyzing a biological sample, the kit comprising: (a) a plurality of nucleic acid probes, wherein each nucleic acid probe of the plurality comprises: (i) a target binding region complementary to a sequence of a target nucleic acid in the sample, and (ii) a barcode region comprising a first and second barcode sequence, wherein the first barcode sequence is selected from a set of first barcode sequences, and the second barcode sequence is selected from a set of second barcode sequences, wherein the first barcode sequence and second barcode sequence are separated by a single-stranded sequence; (b) a plurality of first oligonucleotide probes, wherein each first oligonucleotide probe of the plurality comprises a sequence complementary to one of the first barcode sequences of the set of first barcode sequences, and a sequence complementary to the single-stranded sequence; (c) a plurality of second oligonucleotide probes, wherein each second oligonucleotide probe of the plurality comprises a sequence complementary to one of the second barcode sequences of the set of second barcode sequences; and (d) a nuclease, wherein the nuclease does not recognize the single-stranded sequence, and the nuclease is capable of recognizing a double-stranded restriction site created by hybridization of a first oligonucleotide probe to the nucleic acid probe. In some embodiments, the plurality of nucleic acid probes comprises a group of nucleic acid probes A comprising sequences complementary to sequences of a target nucleic acid A, and a group of nucleic acid probes B comprising sequences complementary to sequences of target nucleic acid B. In some embodiments, the nucleic acid probes and/or oligonucleotide probes can be any of the nucleic acid probes or oligonucleotide probes described in Section III.

In some embodiments, each nucleic acid probe of the plurality of nucleic acid probes comprises an anchor binding region. In some embodiments, the anchor binding region is a common sequence for the nucleic acid probes A and nucleic acid probes B. In some embodiments, the kit further comprises an anchor probe, such as any of the anchor probes described in Section III.

In some embodiments, the nuclease is a restriction endonuclease. In some embodiments, the restriction endonuclease recognizes a double-stranded recognition site that is 4, 5, 6, 7, 8, or more base pairs in length. In some embodiments, the double-stranded recognition site is 6, 7, 8, or more base pairs in length. In some embodiments, cleavage by the restriction endonuclease generates blunt ends. In some embodiments, cleavage by the restriction endonuclease generates sticky ends. Exemplary restriction endonucleases include but are not limited to A anaI, Acc16I, AccII, AccBSI, AcvI, AfaI, AfeI, AjiI, AluBI, Aor51HI, BalI, BmgBI, Bsh1235I, BsnI, Bsp68I, BspANI, BspFNI, BsrBI, BssNAI, Bst11071, BstSNI, BsuRI, BtuMI, DinI, DraI, Ecl136II, Eco105I, Eco147I, Eco321, Eco47III, Eco53kI, Eco721, EcoICRI, EcoRV, EgeI, EheI, FspI, HpaI, HpyCH4V, KspAI, MbiI, MluNI, Mox20I, MscI, Msp20I, MssI, MvnI, NaeI, NruI, NsbI, PceI, PdiI, PmaCI, PmeI, PmII, PsiI, PspCI, PvuII, RruI, ScaI, SfoI, SmaI, SnaBI, SrfI, SseBI, SspI, StuI, SwaI, ZraI, ZrmI, AatII, AbsI, Acc65I, AccIII, AcII, AciI, AfIII, AgeI, AhII, Alw44I, Aor13HI, ApaI, ApaLI, AscI, AseI, AsiGI, AsiSI, Asp718I, AspA2I, AspLEI, AsulI, AvrlI, BamHI, BauI, BbvCI, BcII, BcuI, BfaI, BfrI, BgIII, BlnI, BmgT120I, BmtI, Bpu14I, Bsa29I, BseAI, BseCI, BsePI, BseX3I, BseYI, B shTI, BshVI, BsiWI, BspACI, Bsp119I, Bsp120I, Bsp13I, Bsp1407I, Bsp19I, BspDI, BspEI, BspHI, BspMAI, BspOI, BspT104I, BspTI, BsrGI, BssHII, BssMI, BssSI-v2, Bst2BI, BstAFI, BstAUI, BstBI, BstHHI, BstMBI, BstZI, Bsu15I, BsuTUI CciI, CciNI, CfoI, Cfr42I, Cfr9I, ClaI, CspAI, Csp6I, CviAII, CviQI, DpnII, EagI, EclXI, EcoRI, EcoT22I, FaeI, FatI, FauNDI, FbaI, FseI, GsaI, HapII, HhaI, Hin1II, Hin6I, HindIII, HpaI, HpaII, HpySE526I, Hsp92II, KasI, Kpn2I, KpnI, Ksp22I, KspI, Kzo9I, MaeI, MboI, MluCI, MauBI, MfeI, MluI, Mly113I, Mph1103I, MreI, MroI, MroNI, MspCI, MseI, MspI, MunI, NarI, NcoI, NdeI, NdelI, NgoMIV, NheI, NlaIII, NotI, NsiI, NspV, PaeI, PaeR7I, PagI, PalAI, PauI, PciI, Pfl23II, PinAI, Ple19I, PluTI, PscI, PshBI, Psp124BI, Psp1406I, PspFI, PspLI, PspOMI, PstI, PteI, PvuI, RgaI, RigI, RsaNI, SacI, SacII, SaII, SaqAI, Sau3AI, SbfI, SdaI, SfaAI, Sfr274I, Sfr202I, SfuI, SgfI, SgrBI, SgrDI, SgsI, SlaI, SpeI, SphI, Sse83871, Sse9I, SsiI, SspDI, SspMI, SstI, TaiI, TaqI, TaqI-v2, TasI, TrulI, TspMI, Vha464I, VneI, VspI, XbaI, XhoI, XmaI, and Zsp2I. In some embodiments, the restriction endonuclease is capable of recognizing double-stranded recognition sites with variable or degenerate sequence base positions. Examples of restriction endonucleases recognizing double-stranded recognition sites with variable sequence base positions include AasI, Acc36I, AccB7I, AcIWI, AdeI, AffiI, AhdI, Alw26I, AlwI, AlwNI, Asp700I, AspS9I, AxyI, BbsI, BccI, BcoDI, BfuAI, BgII, BlpI, Bme1390I, BmeRI, BmiI, BmrFI, BmrI, BmuI, BoxI, BpiI, Bpu10I, Bpu1102I, BsaBI, BsaJI, Bsc4I, Bse21I, Bse8I, BseDI, BseGI, BseJI, BseLI, BseMI, BsII, BsmAI, BsmBI-v2, BsmI, Bso31I, Bsp1720I, BspLI, BspMI, BspPI, BspQI, BspTNI, BsrDI, BsrI, BssECI, Bst4CI, Bst6I, BstAPI, BstC8I, BstEII, BstENI, BstF5I, BstMAI, BstMWI, BstPAI, BstPI, BstV2I, BstXI, Bsu36I, BtsCI, BtsI-v2, BtsIMutI, BveI, Cac8I, CaiI, Cfr13I, DdeI, DraIII, DrdI, DriI, DseDI, Eam1104I, Eam1105I, EarI, Eco31I, Eco81I, Eco91I, EcoNI, EcoO65I, Esp3I, FauI, Fnu4HI, Fsp4HI, HinfI, Hpy166II, Hpy188I, Hpy188III, Hpy8I, HpyF10VI, HpyF3I, LguI, LmnI, MaeIII, MroXI, MsII, NIaIV, OliI, PaqCI, PciSI, PctI, PdmI, PflFI, PflMI, PfoI, PleI, PpsI, PshAI, PspEI, PspN4I, PspPI, PstNI, PsyI, RseI, SapI, ScrFI, SfiI, SmiMI, TaaI, Tth111I, Van91I, XagI, XcmI, and XmnI.

### VII. Applications

In some aspects, the provided embodiments can be applied in an in situ method of analyzing nucleic acid sequences, such as an in situ transcriptomic analysis, for example from intact tissues or samples in which the spatial information has been preserved. In some aspects, the embodiments can be applied in an imaging or detection method for multiplexed nucleic acid analysis. In some aspects, the provided embodiments can be used to identify or detect regions of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length.

Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Primer Extension

A “primer extension” refers to any method where two nucleic acid sequences (e.g., a constant region from each of two distinct capture probes) become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

(viii) Nucleic Acid Extension

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

### (ix) PCR Amplification

A “PCR amplification” refers to the use of a polymerase chain reaction (PCR) to generate copies of genetic materiaI, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functionaI, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments (e.g., when the PCR amplification amplifies captured DNA), the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, Mass.), and Ampligase™ (available from Epicentre Biotechnologies, Madison, Wis.). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functionaI, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using techniques such as but not limited to “TAQMAN™” or “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### (x) Antibody

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In generaI, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)₂ fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

### (xi) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, a capture probe or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to a feature or to a capture probe associated with a feature. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a feature, capture probe, or composition to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, DapoxyI, DCFH, DHR, DiA (4-Di-16-ASP), DiD (Di1C18(5)), DIDS, Dil (Di1C18(3)), DiO (DiOC18(3)), DiR (Di1C18(7)), Di-4 ANEPPS, Di-8 ANEPPS, DM-NERF (4.5-6.5 pH), DsRed (Red Fluorescent Protein), EBFP, ECFP, EGFP, ELF® -97 alcohoI, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™1/PO-PRO™-1, POPO™3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red) , Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC) , Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

### EXAMPLE

The following examples are included for illustrative purposes only and are not intended to limit the scope of the present disclosure.

### Example 1: Restriction Digest-Based Sequential Decoding

This example describes a method for detection and decoding of target nucleic acids comprising sequential hybridization of oligonucleotide probes to nucleic acid probes (e.g., primary probes hybridized to mRNA molecules in the sample) and nuclease-based cleavage of the hybridized oligonucleotide probes, wherein hybridization of an oligonucleotide probe to the nucleic acid probe generates a double-stranded cleavage site. In some aspects, the exemplary method provided herein is useful for combinatorial decoding of barcode sequences in a nucleic acid probe without the need for stripping probes between cycles.

In an example, the target nucleic acid within a biological sample (e.g., a tissue sample on a substrate) is an endogenous RNA (e.g., mRNA). Multiple target nucleic acids can be detected simultaneously using the methods provided herein. For example, the target nucleic acid sequences in the biological sample can be designated mRNA A, mRNA B, mRNA C, and so on (e.g., for detection of 200 or more target nucleic acid sequences for a decoding scheme based on four barcode sequences detected using four distinguishable labels, optionally wherein the absence of a label substitutes for one of the four labels, wherein not all of the 256 possible sequential signal codes are assigned). Each target nucleic acid can be hybridized to multiple nucleic acid probes with the same barcode sequences to increase the signal generated that is associated with each target nucleic acid. In some cases where multiple nucleic acid probes are tiled on the target nucleic acid, each nucleic acid probe for binding the same target nucleic acid may share barcode sequences and have different target binding sequences on the same target nucleic acid.

As shown in FIG. 3, each of the nucleic acid probes can include four barcode sequences (BCs), a target-binding region (e.g., a 20-30 bp target binding region), and optionally, an anchor region (right arm of nucleic acid probes shown in FIG. 3). The barcode sequences of the nucleic acid probes are separated by single-stranded sequences that are single-stranded portions of double-stranded restriction endonuclease cleavage sites.

As shown in FIG. 4, an exemplary barcode sequence can comprise about 15 nucleotides (N, wherein N is any nucleotide selected from A, T, G, or C). For the exemplary nucleic acid probes comprising four barcode sequences, the first, second, third, and fourth barcode sequences are selected from first, second, third, and fourth sets of barcode sequences, respectively. The sets of barcode sequences can be designed such that oligonucleotide probes that complementary to a hybridization region comprising a barcode sequence of one set do not cross-react (i.e., hybridize) with a hybridization region comprising a barcode sequence of any of the other sets. In this way, a first, second, third, and fourth plurality of oligonucleotide probes can be designed to hybridize specifically to barcode sequences of the first, second, third, and fourth sets, respectively. In an example, hybridization of the oligonucleotide probes only to barcode sequences of a particular set avoids premature cleavage releasing a subsequent barcode sequence of the nucleic acid probe.

As shown in FIG. 3, the nucleic acid probe can be a U-shaped probe, comprising from 5′ end to 3′ end or from 3′ end to 5′ end, a first barcode sequence, a first single-stranded sequence, a second barcode sequence, a second single-stranded sequence, a third barcode sequence, a third single stranded sequence, a fourth barcode sequence, a fourth single-stranded sequence, a target-binding region, and an anchor binding region. A partial sequence of an exemplary nucleic acid probe is depicted in FIG. 4, wherein the restriction endonuclease is EcoRV.

The biological sample is first contacted with a plurality of nucleic acid probes A, B, C, and so on that hybridize directly or indirectly to the target nucleic acids A, B, C, . . . , and so on, up to the coding capacity of the system as determined by the number of sequential hybridizations, the number of different detectable moieties associated with the oligonucleotide probes, and the number of detection channels of the imaging device. The nucleic acid probes can be designed to tile their respective target nucleic acids (e.g., 20-50 nucleic acid probes hybridized per target).

Next, the sample is contacted with a first plurality of first oligonucleotide probes under conditions that promote hybridization of the first oligonucleotide probes to hybridization regions of the nucleic acid probes comprising complementary first barcode sequences. Each of the first oligonucleotide probes is associated with a first fluorescent moiety or the absence of a fluorescent moiety. The sample is imaged, and the color of the fluorescent moiety at each target nucleic acid position in the sample detected is recorded. The sample is then incubated with a restriction endonuclease under conditions for restriction endonuclease digestion of the double-stranded recognition sites generated by hybridization of the first oligonucleotide probes to the nucleic acid probes. Restriction endonuclease digestion releases a cleaved sequence comprising a portion of the oligonucleotide probe associated with the first barcode sequence. The cleaved sequence can be removed in a wash step. Optionally, a remaining fragment of the oligonucleotide probe can also be removed in the wash step, as the melting temperature of the short (e.g., 3 nucleotide) fragment is substantially lower than the melting temperature of the nucleic acid probes hybridized to the target nucleic acids.

These steps of oligonucleotide probe hybridization, imaging, and restriction endonuclease cleavage can be repeated for the subsequent barcode sequences in order. If oligonucleotide probes lacking fluorescent labels are included as shown in FIG. 3, hybridization of an oligonucleotide probe lacking a label at a given position and in a given cycle can be inferred and recorded based on the detection of labelled oligonucleotide probes at the given position in other cycles.

The biological sample is then optionally contacted with an anchor probe associated with a fluorescent moiety, wherein the anchor probe hybridizes to the anchor binding region of the nucleic acid probes, which can be a common sequence across a plurality of analytes. The sample is then imaged to detect the plurality of analytes simultaneously. In some embodiments, the hybridization and detection of the anchor probe can be performed before or during the cycle of steps for oligonucleotide probe hybridization, imaging, and restriction endonuclease cleavage.

The cycles of contacting, imaging and cleaving can yield sequential signatures of fluorescent signals that form sequential signal codes. The codes (e.g., color of label or absence of label) assigned to the spots from each cycle can be overlaid to generate unique sequential signal codes corresponding to the identity of a given target nucleic acid sequence at a given position in the sample.

It will be understood that variations of the described method can also be performed. For example, the oligonucleotide probes can be L- or U-shaped probes as shown in FIG. 2A, wherein one or more detection probes are hybridized to the L- or U-shaped probes. In this example, the number of different detection probes can correspond to the number of different fluorescent moieties used (e.g., four distinct labelled probes for four channels), rather than the number of distinct barcode sequences in the nucleic acid probes (e.g., 16 distinct barcode sequences for a first, second, third, and fourth set of barcode sequences, wherein each set of barcode sequences corresponds to four different oligonucleotide probes).

In another variation, hybridization of one or more detection probes to an oligonucleotide probe generates a double-stranded recognition site, as shown in FIG. 2B. The restriction enzyme can thus cleave a portion of the oligonucleotide probe associated with the detection probe labelled probe, leaving the barcode sequence in the nucleic acid probe. In such instances, the barcode sequences can be arranged in any order in the nucleic acid probe, as the restriction endonuclease cleavage does not result in the release of barcode sequences.

FIG. 5 shows a variation of the example shown in FIG. 3, where oligonucleotide probes are selectively cleaved based on the unique double-stranded recognition site generated by hybridization of the oligonucleotide probe to the nucleic acid probe. In the example of FIG. 5, the sample is contacted with a first plurality of first oligonucleotide probes under conditions that promote hybridization of the first oligonucleotide probes to hybridization regions of the nucleic acid probes comprising complementary first barcode sequences. Each of the first oligonucleotide probes is associated with a first fluorescent moiety or the absence of a fluorescent moiety. The sample is imaged, and the color of the fluorescent moiety at each target nucleic acid position in the sample detected is recorded, which is depicted in FIG. 5 as “before selective cleavage” for cycle 1. The sample is then incubated with a restriction endonuclease under conditions for restriction endonuclease digestion of double-stranded recognition sites generated by hybridization of the first oligonucleotide probes to the nucleic acid probes that are recognized by the particular restriction endonuclease. For instance, as shown in FIG. 5, the double-stranded recognition sites generated by hybridization of the first oligonucleotide probes to the nucleic acid probes on target nucleic acid A and target nucleic acid C are recognized and thus cleaved by the restriction endonuclease that is incubated with the sample during cycle 1, whereas the double-stranded recognition site generated by hybridization of the first oligonucleotide probes to the nucleic acid probes on target nucleic acid B is not recognized and thus not cleaved by the restriction endonuclease that is incubated with the sample during cycle 1. Restriction endonuclease digestion releases a cleaved sequence comprising a portion of the oligonucleotide probe associated with the first barcode sequence for target nucleic acids A and C during cycle 1, as shown in FIG. 5 as “after selective cleavage.” The cleaved sequence can be removed in a wash step. Optionally, a remaining fragment of the oligonucleotide probe can also be removed in the wash step, as the melting temperature of the short (e.g., 3 nucleotide) fragment is substantially lower than the melting temperature of the nucleic acid probes hybridized to the target nucleic acids. The sample is then imaged, and the color of the fluorescent moiety at each target nucleic acid position in the sample detected is recorded. Further, in some examples, one or more additional steps of selective cleavage can be performed, such as by using a restriction endonuclease to cleave a double-stranded recognition site generated by hybridization of the first oligonucleotide probes to the nucleic acid probes on a target nucleic acid that was not cleaved in a prior selective cleavage step, such as target nucleic acid B in FIG. 5.

These steps of oligonucleotide probe hybridization, imaging, selective restriction endonuclease cleavage, and imaging can be repeated for the subsequent barcode sequences in order, such as shown in FIG. 5 with cycle 2, and this can further include one or more additional cycles.

As with other examples, the cycles of contacting, imaging and selective cleaving can yield sequential signatures of fluorescent signals that form sequential signal codes. The codes (e.g., color of label or absence of label) assigned to the spots from each cycle can be overlaid to generate unique sequential signal codes corresponding to the identity of a given target nucleic acid sequence at a given position in the sample. Selectively cleaving oligonucleotide probes during each cycle allows for generating additional sequential signature of fluorescent signals during each cycle. For instance, as shown in FIG. 5, four different signatures of fluorescent signals are generated using a first and a second plurality of oligonucleotide probes during two cycles.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-105. (canceled)

106. A method of analyzing a biological sample, comprising:
a) contacting the biological sample with a nucleic acid probe, wherein the nucleic acid probe comprises a barcode region comprising one or more barcode sequences, and wherein the nucleic acid probe hybridizes to a target nucleic acid in the biological sample;
b) hybridizing an oligonucleotide probe to a first hybridization region of the nucleic acid probe in the sample;
wherein the first hybridization region comprises a first barcode sequence of the barcode region and a first single-stranded sequence,
wherein hybridization of the oligonucleotide probe to the first single-stranded sequence creates a double stranded recognition site that is recognized by a nuclease, and
wherein the first single-stranded sequence is not recognized by the nuclease;
c) detecting a signal or absence thereof associated with the oligonucleotide probe at one or more locations in the biological sample; and
d) cleaving the double stranded recognition site using the nuclease, thereby releasing a cleaved sequence associated with the oligonucleotide probe.

107. The method of claim 106, wherein the oligonucleotide probe is a first oligonucleotide probe and the nucleic acid probe comprises a second hybridization region comprising a second barcode sequence of the barcode region, and the method further comprises:
contacting the biological sample with a second oligonucleotide probe comprising a sequence complementary to the second barcode sequence of the barcode region, wherein the second oligonucleotide probe hybridizes to the second hybridization region of the nucleic acid probe, and
detecting a signal or absence thereof associated with the second oligonucleotide probe.

108. The method of claim 107, wherein the second hybridization region comprises a second single-stranded sequence,
wherein hybridization of the second oligonucleotide probe to the second single-stranded sequence creates a second double stranded recognition site that is recognized by a nuclease, and
wherein the second single-stranded sequence is not recognized by the nuclease.

109. The method of claim 108, wherein the first and second single-stranded sequences have the same sequence.

110. The method of claim 108, wherein the double stranded recognition site is a first double stranded recognition site, and the first double stranded recognition site and the second double stranded recognition site each comprise a recognition site that is cleaved by a different nuclease.

111. The method of claim 108, wherein the method further comprises cleaving the second double-stranded recognition site using the nuclease, thereby releasing a cleaved sequence associated with the second oligonucleotide probe.

112. The method of claim 107, wherein the nucleic acid probe further comprises a third hybridization region comprising a third barcode sequence of the barcode region, and the method further comprises:
contacting the biological sample with a third oligonucleotide probe comprising a sequence complementary to the third barcode sequence of the barcode region, wherein the third oligonucleotide probe hybridizes to the third hybridization region of the nucleic acid probe, and
detecting a signal or absence thereof associated with the third oligonucleotide probe.

113. The method of claim 106, wherein the first barcode sequence is selected from a first set of different first barcode sequences, and the method comprises contacting the sample with a plurality of first oligonucleotide probes, wherein each first oligonucleotide probe comprises a sequence complementary to one of the first barcode sequences.

114. The method of claim 113, wherein each different barcode sequence within a given set corresponds to a different detectable label or absence thereof.

115. The method of claim 113, wherein the label or absence thereof is directly or indirectly linked to the oligonucleotide probe that hybridizes to the barcode sequence.

116. The method of claim 113, wherein none of the plurality of first oligonucleotide probes hybridize to the any of the second barcode sequences.

117. The method of claim 107, wherein the sequential combination of the detected signals or absence thereof corresponding to the barcode sequences of the barcode region forms a sequential signal code that identifies the target nucleic acid hybridized by the nucleic acid probe.

118. The method of claim 106, wherein the nucleic acid probe or each nucleic acid probe of the plurality of nucleic acid probes comprises two, three, four, five, six, or more barcode sequences.

119. The method of claim 106, wherein the nucleic acid probe comprises two or more single-stranded sequences that each have different sequences.

120. The method of claim 107, wherein the hybridization regions are ordered in the nucleic acid probe such that nuclease cleavage of a double-stranded recognition site created by hybridization of an oligonucleotide probe to a hybridization region does not release a subsequent hybridization region from the nucleic acid probe hybridized directly or indirectly to the target nucleic acid.

121. The method of claim 106, wherein the method comprises removing the cleaved sequence from the biological sample.

122. The method of claim 106, wherein:
the target nucleic acid comprises RNA; or
the target nucleic acid is a rolling circle amplification (RCA) product in the biological sample or in a matrix embedding the biological sample or molecules thereof.

123. The method of claim 106, wherein the nuclease is a restriction endonuclease.

124. A method of analyzing a biological sample, comprising:
a) contacting the biological sample with a plurality of first oligonucleotide probes comprising first oligonucleotide probes A and B,
wherein the biological sample comprises (i) a target nucleic acid A and a target nucleic acid B, and (ii) one or more nucleic acid probes A hybridized to the target nucleic acid A, and one or more nucleic acid probes B hybridized to the target nucleic acid B,
wherein the one or more nucleic acid probes A comprise a barcode region A comprising a first barcode sequence selected from a set of first barcode sequences and a second barcode sequence selected from a set of second barcode sequences,
wherein the one or more nucleic acid probes B comprise a barcode region B comprising a first barcode sequence selected from the set of first barcode sequences and a second barcode sequence selected from the set of second barcode sequences, and
hybridization of the first oligonucleotide probes to the nucleic acid probes A and B creates double stranded recognition sites positioned between the first barcode sequences of barcode regions A and B and the rest of the nucleic acid probes A and B, respectively;
b) at one or more locations in the biological sample, detecting a label or absence thereof associated with the oligonucleotide probes that hybridize to the first barcode sequence of barcode region A and the first barcode sequence of barcode region B;
c) cleaving the double stranded recognition sites using a nuclease, thereby releasing a cleaved sequence associated with the first oligonucleotide probes A and B, respectively, leaving the second barcode sequences of barcode regions A and B in their respective nucleic acid probe;
d) contacting the biological sample with a plurality of second oligonucleotide probes, wherein hybridization of the second oligonucleotide probes to the nucleic acid probes A and B creates double stranded recognition sites positioned between the second barcode sequences of barcode regions A and B and the rest of the nucleic acid probes A and B, respectively;
e) at one or more locations in the biological sample, detecting a label or absence thereof associated with the oligonucleotide probes that hybridize to the second barcode sequences of barcode regions A and B; and
f) using the labels or absence thereof detected in steps b) and e) to generate a sequential signal code corresponding to the target nucleic acid A and the target nucleic acid B, thereby identifying the target nucleic acids A and B at the one or more locations in the biological sample.

125. A method of analyzing a biological sample, comprising:
a) contacting the biological sample with a nucleic acid probe, wherein the nucleic acid probe comprises a barcode region comprising one or more barcode sequences, and wherein the nucleic acid probe hybridizes to a target nucleic acid in the biological sample;
b) hybridizing a first oligonucleotide probe comprising a sequence complementary to a first barcode sequence of the barcode region to the first barcode sequence,
c) hybridizing one or more detection probes to the first oligonucleotide probe,
wherein the detection probes are associated with a label or with the absence of a label,
wherein hybridization of the one or more detection probes to the first oligonucleotide probe creates one or more copies of a double stranded recognition site that is recognized by a nuclease,
d) detecting a signal or absence thereof of the detection probes hybridized to the first oligonucleotide probe;
e) cleaving the double-stranded recognition site using the nuclease, thereby releasing a cleaved sequence associated with the one or more detection probes.
